(19) United States
(12) Patent Application Publication
(10) Pub. No.: US 2019/0390250 A1
(43) Pub. Date: Dec. 26, 2019

(54) FRAGMENTATION OF CHAINS OF NUCLEIC ACIDS

(71) Applicant: THE UNIVERSITY COURT OF THE UNIVERSITY OF GLASGOW, Glasgow (GB)

(72) Inventors: Robert WILSON, Glasgow (GB); Jonathan M. COOPER, Glasgow (GB); Julien REBOUD, Glasgow (GB)

(21) Appl. No.: 16/337,750

(22) PCT Filed: Oct. 10, 2017
(86) PCT No.: PCT/EP2017/075866

§ 371 Date: Mar. 28, 2019

(30) Foreign Application Priority Data
Oct. 10, 2016 (GB) 1617188.6

(51) Int. Cl. C12Q 1/6806; B01L 3/00; G01N 1/42; G01N 29/02

(52) U.S. Cl. CPC C12Q 1/6806; B01L 3/50273; B01L 2400/0436; G01N 29/022; G01N 1/42

(57) ABSTRACT

Disclosed are methods and devices for fragmenting chains of nucleic acids (such as DNA) in a liquid sample. A liquid sample is provided, comprising chains of nucleic acids. A sample treatment device has a sample treatment zone. The liquid sample is contacted with the sample treatment zone. Surface acoustic waves (SAWs) are propagated along a surface of the sample treatment zone, or more generated acoustic waves are propagated to couple with the sample, and/or the sample is subjected to freeze-thaw cycling, in order to cause fragmentation of said chains of nucleic acids in the sample.

### BACKGROUND TO THE INVENTION

### Field of the Invention

The present invention relates to the fragmentation of chains of nucleic acids, such as DNA and/or RNA, using surface acoustic waves (SAWs) or other acoustic waves, and/or using cycles of heating and cooling. The invention has particular, but not exclusive, applicability to the pre-processing of biological samples in preparation for sequencing operations.

### Related Art

Since completion of the first human genome sequence, the demand for cheaper and faster sequencing methods has increased enormously. This need has driven the development of second-generation sequencing methods, or next-generation sequencing (also known as NGS or high throughput sequencing). The technology platform performs massively parallel sequencing, during which millions of fragments of DNA from a single sample are sequenced in unison. Massively parallel sequencing technology facilitates high-throughput sequencing, which allows an entire genome to be sequenced in less than one day. The creation of these platforms has made sequencing accessible to more laboratories, rapidly increasing the volume of research, including clinical diagnostics and its use in directing treatment. For example, in 2014 the Mayo Clinic launched a 50-gene panel to inform drug therapy in a wide range of cancers.

The applications of next generation sequencing are also allowing rapid advances in many clinically related fields in the biological sciences including (i) the re-sequencing of the human genome to identify genes and regulatory elements involved in pathological processes; (ii) comparative biology studies through whole-genome sequencing; (iii) public health and epidemiology through the sequencing of bacterial and viral species to facilitate the identification of novel virulence factors. Such developments illustrate why sequencing is now considered to be the fastest-growing area in genomics (increasing at about 23% per year at the time of writing). The activity is said to be worth $2.5 billion per year at the time of writing, and poised to reach about $9 billion by 2020.

Currently, research institutes and government bodies contribute the largest amount of the end-user market. However, the adoption rate in hospitals is set to increase in the near future, due in part to improvements in the cost-effectiveness of sequencing as well as the increasing number of validated applications for diagnostics. In addition, as the public perception of genetic testing becomes more acceptable, and related ethical concerns diminish, so there is a predicted increase in the use of genetic information in diagnostic and therapeutic activities.

A number of pre-sequencing steps are required to be carried out on a sample, prior to the sequencing reactions. These pre-sequencing steps include the fragmentation of the DNA into smaller sizes for processing, size selection, library preparation and target enrichment.

It is widely acknowledged that the step of DNA fragmentation is the most important technological bottleneck in the pre-sequencing steps carried out on the sample. Cutting of DNA molecules into sizes below 1 kbp (depending on the specific sequencing tool and kit) is typically performed either mechanically or biochemically (through enzymatic reactions). Known approaches to DNA fragmentation are summarised below.

Methods of fragmenting DNA have included the use of enzymes, salts, nebulisation, pumping through small apertures in microfluidic devices and ball milling. At present, mechanical shearing of DNA is preferred, since the enzymatic fragmentation tends to introduce a sequence bias which significantly reduces the quality of the sequencing data downstream [Marine R. et al. (2011)].

A preferred technique to date has used ultrasound to generate random fragments with mean size around 150 bp to 1000 bp depending on the conditions used and the intended sequencing tool. This size range of fragments considered to be ideal for the use with modern sequencing technologies (NGS).

However, current instrumentation for DNA fragmentation is not only expensive but is not readily automatable. As such, the current instrumentation typically sits as a “stand-alone” instrument, distinct from the sequencers. The nature of the methods developed for DNA fragmentation also requires that the sample volume is relatively large, limiting applications in diagnostics, and indeed making field-based approaches difficult to implement. Sample preparation is mostly carried out by trained personnel prior to sequencing of DNA. The need for trained personnel becomes a bottleneck for throughput of samples to be read and constricts the usage to a relatively small demographic.

Specific documents disclosing ultrasound-based DNA fragmentation are briefly discussed below.

WO 93/03150 discloses the use of noninvasive ultrasonication for cell lysis and for genomic DNA fragmentation and denaturation in the same step. Denaturation is promoted by the use of chaotropic agents. The result is stated to be single stranded nucleic acid fragments which are of substantially the same length. The fragments are 400-600 bp in length.

U.S. Pat. No. 6,719,449 discloses ultrasonic techniques including operation in the MHz region, for various applications including cell lysis. The generation of the ultrasound is carefully controlled in order to limit thermal effects in the sample. Cavitation is encouraged in some applications. There is discussion of the application of the technique to DNA, but this is in the context of the sonication technique being used to drive rapid heating and cooling cycles, rather than DNA fragmentation.

US 2008/0031094 discloses apparatus for treating various biological materials, including nucleic acids, using high frequency ultrasonic waves (100 kHz-100 MHz) with a pressurized sample.

US 2009/0233814 discloses DNA fragmentation using ultrasonication of samples of the DNA in combination with particles such as SiC beads.

US 2012/0264228 discloses DNA fragmentation using ultrasound at operating frequencies in the range 28-80 kHz. Again, this document links the use of ultrasound for cell lysis with the use of ultrasound for DNA fragmentation. One of the aims of US 2012/0264228 is to decrease the distribution of the length of the DNA fragments.

US 2013/0092524 discloses DNA fragmentation using directed and steerable ultrasound. A frequency of 4 MHz is used in burst mode to avoid unwanted heating of the sample. The sample is held in a container coupled to the transducer via a coupling medium. The directionality of the ultrasound is provided by the design of the transducer. The aim of US 2013/0092524 is to provide a tight size distribution of DNA fragments.

US 2014/0193305 discloses a cartridge-based approach to DNA fragmentation. The aim is to bring together the sample preparation steps and the sequencing and analysis steps into one apparatus. The cartridge has a planar shape with substantially equi-axed microfluidic sample compartment. This is coupled to an ultrasonic transducer by a fluid coupling medium.

WO 2014/055832 discloses an approach for cell lysis, DNA fragmentation and tissue dispersion, in which encapsulated microbubbles (1-10 micron diameter) are added to a sample before sonication at 0.01-10 MHz. The microbubbles cause cavitation by oscillation or bursting due to the application of ultrasound.

Tseng et al (2012) disclose a sub-microliter microfluidic device for DNA fragmentation using acoustic cavitation driven by a Langevin-type composite transducer operating at 63 kHz. The transducer used by Tseng et al (2012) is bulky.

Okabe and Lee (2014) disclose the use of lateral cavity acoustic transducers for DNA fragmentation. Sample size was 10 μL or less. The ultrasound was generated at about 50 kHz.

Larguinho et al (2010) evaluated several ultrasound-based platforms for DNA sample preparation. The authors recommend a 100 μL sample volume with a DNA concentration of 100 μg/L. Sonication was carried out using a sonoreactor operating at 24 kHz for 2 minutes.

Nama et al (2014) disclose the effect of sharp edges on acoustic streaming, in contrast to the effects seen when mixing is carried out using acoustically driven oscillating bubbles.

### SUMMARY OF THE INVENTION

The present inventors have found that, surprisingly, surface acoustic waves (SAWs, e.g. Rayleigh waves, Lamb waves, shallow bulk acoustic wave (SBAW), surface skimming bulk waves (SSBW) or Hybrid acoustic waves) can drive streaming in a liquid sample with the effect that useful fragmentation of chains of nucleic acids can be obtained.

The present inventors and co-workers have actively researched the field of SAW microfluidics for several years. The present invention builds upon work disclosed in WO 2011/060369, WO 2012/114076, WO 2012/156755 and PCT/GB2014052672 (not yet published at the time of writing). The insight of the inventors has allowed them to develop the present invention, which they consider to provide several practical advantages compared with ultrasound-based fragmentation techniques.

Taking the disclosure of US 2014/0193305 as an example, in that document there is a proposal to subject a microfluidic cartridge to ultrasonic excitation, in order to promote DNA fragmentation in a liquid sample contained in the cartridge. This approach has the useful effect of allowing interfacing of the treated sample with a sequencing apparatus via an automated procedure. However, the present inventors consider that the approach to DNA fragmentation in US 2014/0193305 is susceptible of further improvement, in particular in terms of increasing the efficiency of fragmentation, in order to avoid deleterious and unwanted heating of the sample during treatment.

The present inventors also recognize that further automation of fragmentation techniques would improve the work flow for sequencing, in order to make the process more time efficient and less costly. This would open up opportunities to a wider group of users from individual citizens to developing world countries. The present inventors also recognize that enabling a planar system topology would make it easier to implement a simpler work flow and compact device architecture which can be the basis of a portable system. Further, the present inventors have realised that for some preferred implementations, the utilization of surface waves is advantageous when working with small volume samples because a greater proportion of the sample can then be exposed to the energy in the wave. Such an advantage is consistent with chip based technologies.

The present invention has therefore been devised in order to address at least one of the above problems. Preferably, the present invention reduces, ameliorates, avoids or overcomes at least one of the above problems.

In a first general aspect, therefore, the present inventors propose to use SAWs to drive fragmentation in a liquid sample. It is considered that using SAWs allows fragmentation to take place more efficiently and with reduced heating compared with known ultrasonic approaches, because the coupling of SAWs into the sample is considered to be an interface effect.

Accordingly, in a first preferred aspect, the present invention provides a method of fragmenting chains of nucleic acids in a liquid sample, the method including the steps:

providing a liquid sample comprising chains of nucleic acids;

providing a sample treatment device, the sample treatment device having a sample treatment zone;

contacting said sample with said sample treatment zone; and

generating and propagating surface acoustic waves (SAWs) along a surface of the sample treatment zone, said SAWs coupling into the sample to cause fragmentation of said chains of nucleic acids in the sample.

The liquid sample may have volume V, and an area of an interface between the sample and the sample treatment zone may be area A. Preferably, the ratio A/V is at least 1000 m²/m³. It is recognised that the units m²/m³ may be unwieldy when dealing with volumes in the μL range, but in view of the comparison being made between area and volume, these units are chosen for the sake of certainty. Working in this range of ratio A/V provides efficient fragmentation. It is considered that this range ensures high interfacial surface area at which SAWs can couple into the liquid sample compared with the volume of the sample. This allows efficient fragmentation whilst reducing unwanted sample heating. More preferably, ratio A/V is at least 1200 m²/m³, more preferably at least 1400 m²/m³, more preferably at least 1600 m²/m³. For practical purposes, preferably the ratio A/V is at most 10000 m²/m³.

The sample treatment zone may include an area having roughness Rz at least 10 μm. It is considered that such surface roughness is of use in pinning the liquid sample at the sample treatment zone. Additionally, the surface roughness promotes the available area for coupling of the SAWs into the sample. The surface roughness may be ordered or non-ordered.

The sample treatment zone may include an array of cavities, being ordered or non-ordered, the cavities cumulatively containing at least part of the sample, optionally all of the sample. The cavities are considered to provide a similar effect to the surface roughness introduced above.

The sample treatment zone may include an array of pillars, being ordered or non-ordered. The pillars are considered to provide a similar effect to the surface roughness introduced above.

Preferably, the contact angle between the sample and the sample treatment zone is lower than between the sample and a remaining part of the SAW transmission surface, in order to locate the sample.

Preferably, the sample has a volume of not more than 30 μL, more preferably not more than 15 μL. This is a small sample volume. Typical ultrasound fragmentation techniques use substantially greater volumes. The present invention is therefore particularly advantageous when large sample volumes are not available. It has been found that the invention works satisfactorily even at lower sample volumes, e.g. not more than 10 μL, such as at about 5 μL.

Preferably, the concentration of the chains of nucleic acids in the sample is in the range 5-100 ng/μL. The invention has particular advantages at relatively low concentrations, for example in the range 5-50 ng/μL, because this allows the sample pre-processing to be relatively gentle.

The SAW transmission surface may be a surface of the SAW transducer. However, more preferably, the SAW transmission surface is a surface of a superstrate coupled to the SAW transducer.

The present invention is not necessarily limited to any particular orientation. The term “superstrate” is used because in typical implementations of embodiments of the invention, this item is placed on top of the SAW transducer. However, other orientations are contemplated, e.g. in which a corresponding substrate is placed under the transducer, yet the same effect of the invention can be seen, in which chains of nucleic acids in the sample are fragmented.

Furthermore, the present invention is not necessarily limited to a planar configuration, although a planar configuration may have particular advantages for interoperability with a sequencer, as explained in more detail below. Where a configuration other than a planar configuration is used, for example, the transducer may be formed inside the superstrate, e.g. in a tubular configuration. Alternatively, the transducer may be formed around the superstrate, with the superstrate in the form of a tube (or hollow needle) held inside a transducer tube. This may be preferred, in order that a continuous (or quasi continuous) supply of sample fluid may be provided to the superstrate tube, for continuous fragmentation.

Preferably, the superstrate is formed of a material which is impervious to the liquid. This helps to avoid any (potentially contaminating) contact between the transducer and the liquid.

Preferably, the transducer comprises a layer of piezoelectric material. For example, the layer of piezoelectric material may be a sheet (e.g. a self-supporting sheet) of piezoelectric material. The layer of piezoelectric material may be a single crystal, such as a single crystal wafer. A suitable material is LiNbO₃. A preferred orientation for the cut for this material is Y-cut rot. 128°. This has a higher electromechanical coupling coefficient than other orientations. Other ferroelectric materials may be used, e.g. PZT, BaTiO₃, SbTiO₃ or ZnO. Still further, materials such as SiO₂ (quartz), AlN, LiTaO₃, Al₂O₃GaAs, SiC or polyvinylidene fluoride (PVDF) may be used. As an alternative to a single crystal, the material can be provided in polycrystalline or even amorphous form, e.g. in the form of a layer, plate or film.

The transducer preferably further comprises at least one arrangement of electrodes. For example, the electrodes may be interdigitated. More preferably, the transducer comprises two or more arrangements of electrodes. In some embodiments, it is preferred that the transducer is tunable, such that the lateral position of the SAWs emission train is movable. For example, the slanted interdigitated arrangement of electrodes suggested by Wu and Chang (2005) can be used for the transducer.

The superstrate may be permanently coupled to the piezoelectric layer, in the sense that it is not removable from the piezoelectric layer without damage to the device.

Alternatively, coupling between the transducer and the superstrate may be achieved using a coupling medium, preferably a fluid or gel coupling medium. The coupling medium may be an aqueous coupling medium, e.g. water. Alternatively, the coupling medium may be an organic coupling medium, such as an oil-based coupling medium or glycerol. The coupling medium provides intimate contact between the superstrate and the transducer and allows the efficient transfer of acoustic energy to the superstrate from the transducer.

The advantage of providing the superstrate as a separate entity from the transducer is very significant. Typical SAW transducers are complex to manufacture. For this reason, they are typically expensive. Contamination of the transducer may be difficult or impossible to remove, if the liquid is allowed to come into contact with the transducer. Alternatively, removal may not be cost-effective, or may damage the transducer. However, it is strongly preferred that the transducer can be re-used. Accordingly, it is preferred that the liquid does not contact the transducer but instead contacts the superstrate coupled to the transducer. The superstrate itself may be disposable (e.g. disposed of after a single use). The superstrate may be formed by various methods, such as microfabrication, embossing, moulding, spraying, lithographic techniques (e.g. photolithography), etc.

Where cavities are present, they may have substantially the same shape. The SAW transmission surface, in use, preferably is held substantially horizontal. In this way, the cavities preferably open in the upward direction. The cavities may be substantially columnar in shape. In this way, the cross sectional shape of the cavities may be substantially uniform with depth (a direction perpendicular to the SAW transmission surface). For example, the cross sectional shape of the cavities in the depth direction may be rectangular, square, rounded, oval, elliptical, circular, triangular. Most preferably the cross sectional shape of the cavities in the depth direction is circular. The cross sectional area of the cavities may be uniform with depth. However, in some embodiments this may not be the case, allowing the cavities to have a cross sectional area which narrows, expands or undulates with depth. For example, funnel-shaped cavities may be provided (such cavities being capable of being formed using a KOH etch for example), to provide suitable volume in the cavity to retain the liquid.

The cavities may have an internal structure. For example, there may be provided one or more pillars upstanding in the cavities, walls projecting into the cavities or other projections into the cavities. The internal walls of the cavities may have one or more array of such projections. The array of projections may be considered to be a phononic structure, in the sense that it is based on a periodic arrangement (in the manner disclosed in WO 2011023949, WO 2011060369, WO 2012114076 and WO 2012156755) for affecting the distribution and/or transmission of SAWs in the cavities.

Such internal structures increase the interfacial surface area A2 (see below) between the sample treatment zone and the sample in a manner which can further improve the performance of the device in fragmenting DNA.

The cavities preferably have substantially the same dimensions.

Preferably the depth of the cavities is at least 1 μm. Preferably the depth of the cavities is at most 1 mm, more preferably at most 500 μm.

Preferably the maximum dimension of the cavities in a direction perpendicular to the depth of the cavities is at least 1 μm. The lower limit may be at least 2 μm, at least 5 μm, at least 10 μm, at least 20 μm, at least 30 μm, at least 40 μm or at least 50 μm. Preferably, this maximum dimension is at most 500 μm, more preferably at most 400 μm, at most 300 μm or at most 200 μm. Where the cavities have a circular cross section shape, this dimension is referred to as the diameter of the cavities. Where the cavities have a non-circular cross sectional shape, this maximum dimension is also referred to as the diameter.

The cavities may contain the liquid sample so that each cavity contains a discrete volume of the sample, without a liquid path between the cavities. In this way, when the sample treatment zone is oriented horizontally, the upper surface of the sample in each cavity may be below the top of each cavity. Alternatively, the liquid sample may be only partially contained in the cavities, so that the upper surface of the liquid sample is above the top of each cavity, with a liquid path between the filled cavities.

As mentioned above, preferably the cavities have substantially the same dimensions. However, it is allowable for the cavities to have a distribution of dimensions. In terms of the diameter of the cavities, preferably the standard deviation of the diameter is 40% or less, more preferably 30% or less, more preferably 20% or less.

The cavities can be in the form of cylindrical holes. A suitable volume for the cavities can be at least 0.5 nl, more preferably at least 1 nl. This volume is preferably at most 10 nl, more preferably at most 5 nl. As an example, a cylindrical hole of diameter 100 μm and depth 300 μm has a volume of about 2 nl.

The array of cavities may not have long range order. In this case, the arrangement of the cavities may be substantially random, in the sense of not being based on a periodic arrangement.

The frequency of the surface acoustic wave may be in the range of more than 100 kHz to about 1 GHz. More preferably the frequency may be in the range of about 1 MHz to about 50 MHz. Still more preferably the frequency may be in the range of about 1 MHz to about 10 MHz.

The SAW transducer may be formed from any suitable material for generating surface acoustic waves. SAWs may be generated, for example, by a piezoelectric process, by a magnetostrictive process, by an electrostrictive process, by a ferroelectric process, by a pyroelectric process, by a heating process (e.g. using pulsed laser heating) or by an electromagnetic process. It is most preferred that the SAW generation material layer is formed from a piezoelectric layer. In the disclosure set out below, the term “piezoelectric layer” is used but is it understood here that similar considerations would apply to SAW generation material layers formed, for example, of magnetostrictive materials. Therefore, unless the context demands otherwise, the optional features set out in relation to the “piezoelectric layer” are to be understood as applying more generally to the SAW generation material layer, when formed of any suitable material.

The sample treatment zone may be treated in order to promote the containment of the liquid sample at the sample treatment zone. For aqueous liquids, preferably the sample treatment zone is formed to be hydrophilic. Preferably, an area of the SAW transmission surface at which it is not intended for the liquid sample to be located is formed to be hydrophobic, to promote the pinning of the liquid sample at the sample treatment zone.

Preferably, the temperature of the sample is controlled so as not to exceed 45° C., more preferably not to exceed 40° C., more preferably not more than 37° C., still more preferably not more than 20° C. The coupling of SAWs into the liquid sample causes heating, but this in turn risks damage to the nucleic acid fragments. The temperature is linked to the weakening of the double helix which is sequence specific (that is, some sequences melt before others, creating pockets of weakness), thus creating bias in the fragmentation, which is preferably avoided. Therefore control of the temperature is important to ensure that the fragmentation does as little damage to the nucleic acid fragments as possible, whilst still providing useful fragment lengths.

Preferably, the sample is subjected to active cooling. The sample may be frozen, or partially frozen, before the start of coupling SAWs into the sample. This has a surprising beneficial effect, possibly in view of the effect of the rough ice-liquid water interface in the sample. This is discussed in more detail below.

Preferably, the duty cycle of the SAW generation is controlled in order to control the temperature of the sample.

As will be understood, a further advantage of the ratio A/V used in preferred embodiments of the present invention is to allow efficient cooling of the sample.

The surface area A is preferably determined as the footprint area of the sample on the sample treatment zone, viewed in plan view.

In the case for example of the sample treatment zone being open to allow loss of sample due to nebulization, then preferably loss of sample due to nebulization is controlled to be less than 1%.

The power transmitted to the sample can be determined, for example, using a power meter to measure the forward power and the reflected power from the transducer which generates the acoustic waves. The difference between the forward and reflected power is taken to be the power transmitted to the sample. Preferably, the power transmitted to the sample is less than 10 W. More preferably, the power transmitted to the sample is not greater than 8 W, not greater than 6 W or not greater than 4 W. Using such low powers, the present invention provides substantial advantages over ultrasonic-based prior art disclosures, in which the high transmitted powers risk thermal damage to the DNA. Additionally, the use of these low powers permits the system to be implements in a portable device and/or integrated into existing technologies for DNA sequencing.

For devices in which the sample treatment zone has a structured surface for contact with the sample, slightly higher powers may be used, e.g. at least 5 W and up to 18 W. However, it is still possible to use the power ranges identified above, particularly if the power is transmitted in continuous mode.

Preferably, the device includes an active cooling means in thermal contact with the sample treatment zone.

In the foregoing discussion, it is explained that in some cases a non-planar interface between the sample treatment zone and the sample may be advantageous. It is considered that these advantages relate to the efficient coupling of the acoustic waves into the sample and also to the temperature control of the sample. However, these effects are not necessarily limited to the situation where the acoustic waves are SAWs.

Accordingly, in a second aspect, the present invention provides a method of fragmenting chains of nucleic acids in a liquid sample, the method including the steps: providing a liquid sample comprising chains of nucleic acids;

providing a sample treatment device, the sample treatment device having a sample treatment zone;

contacting said sample with said sample treatment zone;

generating and propagating acoustic waves in the sample treatment device;

coupling said acoustic waves into the sample to cause fragmentation of said chains of nucleic acids in the sample,

wherein:

at the sample treatment zone, there is provided a reference surface and at least one sample treatment structure formed in relief from the reference surface so that a surface of the sample treatment structure is disposed at a distance of at least 10 μm from the reference surface.

Optional features set out with respect to the first aspect may be applied in any combination with the second aspect, and vice versa, unless the context demands otherwise.

Preferably, there is provided an array of sample treatment structures at the sample treatment zone. For example, the sample treatment structures may be in the form of an array of pillars upstanding from the reference surface of the sample treatment zone. Alternatively, the sample treatment structures may be in the form of an array of troughs recessed from the reference surface of the sample treatment zone. In that case, preferably the troughs are aligned substantially parallel with respect to the wavefronts of the propagating acoustic waves.

The sample treatment structures may be in the form of an array of strips upstanding from the reference surface of the sample treatment zone. In that case, preferably the strips are aligned substantially parallel with respect to the wavefronts of the propagating acoustic waves.

The surface of the sample treatment structure may be substantially parallel to the reference surface. A side wall of the sample treatment structure typically extends between the surface of the sample treatment structure and the reference surface. The side wall meets the reference surface at a joining portion to define a radius of curvature at the joining portion in a plane perpendicular to the reference surface, this radius of curvature preferably being not more than 5 μm. Thus, the joining portion is relatively sharp. It is considered that this assists in the fragmentation mechanism.

When the side wall of the sample treatment structure extends between the surface of the sample treatment structure and the reference surface via an overhang, preferably the point of closest approach between the overhang and the reference surface is at least 0.5 times the distance between the reference surface and the surface of the sample treatment structure. This allows the sample to reach the side wall of the sample treatment structure, in order to enhance its role in the fragmentation mechanism.

In a third preferred aspect, the present invention provides a method of fragmenting chains of nucleic acids in a liquid sample, the method including the steps:

providing a liquid sample comprising chains of nucleic acids;

providing a sample treatment device, the sample treatment device having a sample treatment zone;

contacting said sample with said sample treatment zone;

generating and propagating acoustic waves in the sample treatment device;

coupling said acoustic waves into the sample to cause fragmentation of said chains of nucleic acids in the sample,

wherein:

the sample treatment zone is formed with a non-ordered roughness Rz of at least 10 μm.

The sample treatment zone may include an array of cavities, being ordered or non-ordered, the cavities cumulatively containing at least part of the sample, optionally all of the sample.

The sample treatment zone may include an array of pillars, being ordered or non-ordered.

Where cavities are present, they may have substantially the same shape. The sample treatment zone, in use, preferably is held substantially horizontal. In this way, the cavities preferably open in the upward direction. The cavities may be substantially columnar in shape. In this way, the cross sectional shape of the cavities may be substantially uniform with depth. For example, the cross sectional shape of the cavities in the depth direction may be rectangular, square, rounded, oval, elliptical, circular, triangular. Most preferably the cross sectional shape of the cavities in the depth direction is circular. The cross sectional area of the cavities may be uniform with depth. However, in some embodiments this may not be the case, allowing the cavities to have a cross sectional area which narrows, expands or undulates with depth. For example, funnel-shaped cavities may be provided (such cavities being capable of being formed using a KOH etch for example), to provide suitable volume in the cavity to retain the liquid.

The cavities may have an internal structure. For example, there may be provided one or more pillars upstanding in the cavities, walls projecting into the cavities or other projections into the cavities. The internal walls of the cavities may have one or more array of such projections. The array of projections may be considered to be a phononic structure, in the sense that it is based on a periodic arrangement (in the manner disclosed in WO 2011023949, WO 2011060369, WO 2012114076 and WO 2012156755) for affecting the distribution and/or transmission of acoustic waves in the cavities.

Such internal structures increase the interfacial surface area between the sample treatment zone and the sample in a manner which can further improve the performance of the device in fragmenting DNA.

The cavities preferably have substantially the same dimensions.

Preferably the depth of the cavities is at least 1 μm. Preferably the depth of the cavities is at most 1 mm, more preferably at most 500 μm.

Preferably the maximum dimension of the cavities in a direction perpendicular to the depth of the cavities is at least 1 μm. The lower limit may be at least 2 μm, at least 5 μm, at least 10 μm, at least 20 μm, at least 30 μm, at least 40 μm or at least 50 μm. Preferably, this maximum dimension is at most 500 μm, more preferably at most 400 μm, at most 300 μm or at most 200 μm. Where the cavities have a circular cross section shape, this dimension is referred to as the diameter of the cavities. Where the cavities have a non-circular cross sectional shape, this maximum dimension is also referred to as the diameter.

The cavities may contain the liquid sample so that each cavity contains a discrete volume of the sample, without a liquid path between the cavities. In this way, when the sample treatment zone is oriented horizontally, the upper surface of the sample in each cavity may be below the top of each cavity. Alternatively, the liquid sample may be only partially contained in the cavities, so that the upper surface of the liquid sample is above the top of each cavity, with a liquid path between the filled cavities.

As mentioned above, preferably the cavities have substantially the same dimensions. However, it is allowable for the cavities to have a distribution of dimensions. In terms of the diameter of the cavities, preferably the standard deviation of the diameter is 40% or less, more preferably 30% or less, more preferably 20% or less.

The cavities can be in the form of cylindrical holes. A suitable volume for the cavities can be at least 0.5 nl, more preferably at least 1 nl. This volume is preferably at most 10 nl, more preferably at most 5 nl. As an example, a cylindrical hole of diameter 100 μm and depth 300 μm has a volume of about 2 nl.

The array of cavities may not have long range order. In this case, the arrangement of the cavities may be substantially random, in the sense of not being based on a periodic arrangement.

The present inventors have realised that the use of a structured interface at the sample treatment zone allows control over the coupling of acoustic energy into the sample. In some embodiments, the acoustic waves at the sample treatment zone include surface shear waves. For a completely planar sample treatment zone surface, such shear waves would not adequately couple into the liquid sample. However, where coupling projections are provided at the sample treatment zone surface, the shear waves cause the coupling projections to oscillate transversely to the sample treatment zone surface and thereby impart compressional waves into the liquid sample.

Preferably, the coupling projections are provided as longitudinally extending waveguides along or across the sample treatment zone. In this way, the side walls of the longitudinally extending waveguides effectively convey Rayleigh or Lamb waves.

Preferably, in these embodiments, the acoustic waves include Bleustein-Gulyaev waves and/or guided Love waves.

The present inventors have found that temperature control of the sample provides a surprising effect in terms of efficiency of fragmentation of chains of nucleic acids in the sample. This is found to be particularly marked when the sample includes ice crystals during at least part of the time for which the sample is treated. Furthermore, it has been found that this efficacy is demonstrated not only when the sample is treated using SAWs but also more generally when the sample is treated using acoustic waves (e.g. bulk ultrasound waves). This finding constitutes the basis for the second general aspect of the invention.

Accordingly, in a fourth preferred aspect, the present invention provides a method of fragmenting chains of nucleic acids in a liquid sample, the method including the steps: providing a sample comprising chains of nucleic acids;

subjecting the sample to acoustic waves to cause fragmentation of said chains of nucleic acids in the sample,

wherein, for at least part of the time for which the sample is subjected to the acoustic waves, the sample includes ice crystals.

At the time of writing, the mechanism for the improvement in efficiency of fragmentation is not clearly understood. Without wishing to be limited by theory, the inventors speculate that a possible mechanism for the effect seen is that the ice crystals available in the partially melted sample present to the liquid phase a roughened interface. Additionally or alternatively, relatively small ice crystals may be free to move within the liquid phase. These characteristics of the sample may assist with mechanical breaking up of the nucleic acid chains, in particular when in the presence of a harmonic forcing caused by the acoustic waves. An alternative explanation of the phenomenon (not necessary mutually exclusive from the mechanisms mentioned above) is that there are thermodynamic considerations related to repeated cycles of crystallization, thawing and recrystallization, assisting with breaking up of the nucleic acid chains. These mechanisms may operate in combination. Such mechanisms (although not necessarily on the context of DNA fragmentation) are discussed in Shao et al (2010). Shao et al (2010) explain that when the temperature is reduced to the freezing point of water, water molecules rearrange and form hexagonal ice crystals, which expand to occupy a larger volume than water in the liquid state. The formation of ice crystals during freezing and reformation of ice crystals during thawing generates enormous tension forces.

Optional features set out with respect to the first, second and/or third aspect may be applied in any combination with the fourth aspect, and vice versa, unless the context demands otherwise.

Preferably, the temperature of the sample is controlled during the time for which the sample is subjected to the acoustic waves. The acoustic waves couple into the sample and cause heating. The maximum temperature of the sample during this time is preferably 37° C. More preferably, the maximum temperature of the sample during this time is 35° C., 30° C., 25° C., 20° C., 15° C. or 10° C. Even more preferably, the maximum temperature of the sample during this time is 5° C. or 4° C.

At the beginning of the time for which the sample is subjected to the acoustic waves, the sample may be partially frozen. Alternatively the sample may be completely frozen. More generally, preferably the sample is at or close to the triple point of water.

At the beginning of the time for which the sample is subjected to the acoustic waves, the temperature of the sample may be 0° C. or less. For example, the temperature of the sample may be −5° C. or less, more preferably −10° C. or less. For example, the sample may be at about −20° C. Depending on the power of the acoustic waves to which the sample is subjected, and depending on any cooling applied to the sample during the method, the sample typically heats during the time for which the sample is subjected to the acoustic waves. Therefore, at the start of the time for which the sample is subjected to the acoustic waves, the sample may be fully frozen. At the end of the time for which the sample is subjected to the acoustic waves, the sample may be partially melted or fully melted.

The effect of this approach has been found to be that the nucleic acid fragmentation occurs at relatively low applied powers than if ice crystals are not present in the sample at the start of subjecting the sample to acoustic waves for fragmentation. The manner of measuring the applied power is explained above.

In the second, third and fourth aspects of the invention, the acoustic waves are preferably SAWs. SAWs are preferred for their relative ease of generation and their controllability, for example using phononic structures. SAWs, include, for example, Rayleigh waves, Lamb waves, shallow bulk acoustic wave (SBAW), surface skimming bulk waves (SSBW) or Hybrid acoustic waves. However, other acoustic waves are acceptable, separately or in combination. Suitable acoustic waves include lateral waves such as Love waves and/or Bluestein-Gulyaev type waves.

In a similar manner to the first aspect, the method of the second, third or fourth aspect typically includes the step of providing a sample treatment device. The sample treatment device typically has a sample treatment zone for location of the sample. The acoustic waves may be coupled into the sample via the sample treatment zone.

In the following discussion, the means for generating the acoustic waves (whether SAWs or otherwise) is referred to as the transducer. Reference to acoustic waves is intended to include SAWs, unless the context demands otherwise.

When the sample treatment zone is considered as the first sample treatment zone, the device may include an opposing member providing a second sample treatment zone, adapted to be located in contact with the sample opposite the first sample treatment zone, so that the sample is sandwiched between the first and second sample treatment zones, the opposing member being operable to be reciprocated relative to the first sample treatment zone. Preferably, the opposing member reciprocates at a frequency of less than 1 kHz.

The acoustic waves are preferably generated by an acoustic wave transducer. For the first sample treatment zone, preferably the acoustic waves are generated by a first acoustic wave transducer, e.g. a first SAW transducer. For the second sample treatment zone provided at the opposing member, there may be provided a second acoustic wave transducer, e.g. a second SAW transducer. In the case of SAW transducers, preferably each SAW transducer is adapted to generate and propagate SAWs along a respective SAW transmission surface including the respective sample treatment zone, for coupling said SAWs into the sample to cause fragmentation of said chains of nucleic acids in the sample.

During irradiation of the sample with acoustic waves to achieve fragmentation, the sample may present a free surface. However, it is found that it is preferred in some cases to enclose the sample in a sample chamber. This is preferred in particular to reduce or avoid loss of the sample due to nebulisation. The sample chamber may be coupled to the transducer via the walls of the sample chamber, thereby bringing the sample into direct contact with the transducer. Alternatively, the sample chamber may be coupled to the transducer via a superstrate, interposed between the transducer and the sample. In this case, the superstrate may serve additionally as a wall of the sample chamber.

The sample may be contained in the sample chamber with no other material contained in the sample chamber. However, in some embodiments, it is preferred for the sample to be located in the sample chamber with an immiscible phase. For example, the sample may be wholly or partially encapsulated with an immiscible phase. Where the sample is aqueous, for example, the immiscible phase may be oil or wax-based. Encapsulation of the sample in the immiscible phase may be achieved for example by first freezing a droplet of the sample of the required volume, and then encapsulating it in the immiscible phase and placing the composite encapsulated droplet in the sample chamber. This approach has particular benefits in terms of integration with existing technologies for sequencing operations. Additionally, the encapsulant may provide additional surface area for coupling the acoustic waves into the sample.

The inventors have further realised that freezing and thawing of the sample may independently promote the fragmentation of DNA. Thus, it is possible in some embodiments to fragment DNA without the application of SAWs or acoustic waves generally.

Accordingly, in a fifth preferred aspect, the present invention provides a method of fragmenting chains of nucleic acids in a liquid sample, the method including the steps: providing a sample comprising chains of nucleic acids;

providing a sample treatment device, the sample treatment device having a sample treatment zone;

contacting said sample with said sample treatment zone;

heating and cooling the sample at the sample treatment zone to repeatedly melt and freeze at least part of the sample, to promote fragmentation of said chains of nucleic acids in the sample.

Optional features set out with respect to the first, second, third and/or fourth aspect may be applied in any combination with the fifth aspect, and vice versa, unless the context demands otherwise.

The sample may be subjected to at least 5 cycles, at least 10 cycles, at least 20 cycles or at least 40 cycles of melting and freezing. The sample may be subjected to cycles of melting and freezing at a frequency of at least 0.01 Hz, more preferably at least 0.02 Hz, more preferably at least 0.04 Hz, more preferably at least 0.06 Hz, more preferably at least 0.08 Hz, more preferably at least 0.1 Hz.

The temperature of the sample is preferably controlled so that the maximum temperature of the sample during heating and cooling is 37° C., more preferably at most 10° C. The minimum temperature of the sample during heating and cooling is preferably not lower than −20° C., more preferably not lower than −5° C.

The sample treatment zone may include an area having roughness Rz at least 5 μm. The roughness may be non-ordered.

As described for other aspects of the invention, there may be provided one or more sample treatment structure.

In a sixth preferred aspect, the present invention provides a sample treatment device for fragmenting chains of nucleic acids in a liquid sample, the device having:

a surface acoustic wave (SAW) transmission surface having a sample treatment zone; a SAW transducer adapted to generate and propagate SAWs along the SAW transmission surface including the sample treatment zone, for coupling said SAWs into the sample to cause fragmentation of said chains of nucleic acids in the sample;

wherein the sample treatment zone includes an area having a non-ordered roughness Rz of at least 10 μm.

The inventors consider that their insight into the effect of surface roughness also applies to other types of acoustic waves interacting with the liquid sample to cause DNA fragmentation.

Accordingly, in a seventh preferred aspect, the present invention provides a sample treatment device for fragmenting chains of nucleic acids in a liquid sample, the sample treatment device having a sample treatment zone for contacting said sample, a transducer for generating and propagating acoustic waves in the sample treatment device, to couple said acoustic waves into the sample to cause fragmentation of said chains of nucleic acids in the sample, wherein the sample treatment zone includes an area having non-ordered roughness Rz at least 10 μm.

In an eighth preferred aspect, the present invention provides a sample treatment device for fragmenting chains of nucleic acids in a liquid sample, the device having: a surface acoustic wave (SAW) transmission surface having a sample treatment zone; a SAW transducer adapted to generate and propagate SAWs along the SAW transmission surface including the sample treatment zone, for coupling said SAWs into the sample to cause fragmentation of said chains of nucleic acids in the sample; wherein the device includes an active cooling means in thermal contact with the sample treatment zone.

In a ninth preferred aspect, the present invention provides a sample treatment device for fragmenting chains of nucleic acids in a liquid sample, the device having:

a surface acoustic wave (SAW) transmission surface having a sample treatment zone; a SAW transducer adapted to generate and propagate SAWs along the SAW transmission surface including the sample treatment zone, for coupling said SAWs into the sample to cause fragmentation of said chains of nucleic acids in the sample;

wherein when the sample treatment zone is considered as the first sample treatment zone, the device includes an opposing member providing a second sample treatment zone, adapted to be located in contact with the sample opposite the first sample treatment zone, so that the sample is sandwiched between the first and second sample treatment zones, the opposing member being operable to be reciprocated relative to the SAW transmission surface

Preferably, the opposing member reciprocates at a frequency of less than 1 kHz

When the SAW transducer is considered as the first SAW transducer and the SAW transmission surface is considered as the first SAW transmission surface, the opposing member may provide a second SAW transducer adapted to generate and propagate SAWs along a second SAW transmission surface including the second sample treatment zone, for coupling said SAWs into the sample to cause fragmentation of said chains of nucleic acids in the sample.

Preferably, one or more phononic structures are provided in order to affect the SAW distribution at the sample treatment zone.

More generally, in a tenth preferred aspect, the present invention provides a sample treatment device for fragmenting chains of nucleic acids in a liquid sample, the device having:

a sample treatment zone;

an acoustic wave transducer adapted to generate and propagate acoustic waves to the sample treatment zone, for coupling said acoustic waves into the sample to cause fragmentation of said chains of nucleic acids in the sample;

wherein when the sample treatment zone is considered as the first sample treatment zone, the device includes an opposing member providing a second sample treatment zone, adapted to be located in contact with the sample opposite the first sample treatment zone, so that the sample is sandwiched between the first and second sample treatment zones, the opposing member being operable to be reciprocated relative to the sample treatment zone.

Preferably, the opposing member reciprocates at a frequency of less than 1 kHz.

For the first sample treatment zone, preferably the acoustic waves are generated by a first acoustic wave transducer. For the second sample treatment zone provided at the opposing member, there may be provided a second acoustic wave transducer, adapted to generate and propagate acoustic waves to the second sample treatment zone, for coupling said acoustic waves into the sample to cause fragmentation of said chains of nucleic acids in the sample.

In an eleventh preferred aspect, the present invention provides a sample treatment device for fragmenting chains of nucleic acids in a sample, the device having: a sample treatment zone for contacting the sample;

an active cooling means in thermal contact with the sample treatment zone;

an active heating means configured to provide heat to the sample at the sample treatment zone;

the device being operable to heat and cool the sample at the sample treatment zone to repeatedly melt and freeze at least part of the sample, to promote fragmentation of said chains of nucleic acids in the sample.

The active cooling means may be any means suitable to permit re-freezing of at least part of the sample after heating. For example, a cold chamber could be used, or a pre-cooled heat sink.

Optional features set out with respect any other aspect of the invention may be applied to the eleventh aspect, and vice versa, unless the context demands otherwise.

In a twelfth preferred aspect, the present invention provides a method for performing sequencing of chains of nucleic acids, including the steps:

carrying out the method of the first, second, third, fourth or fifth aspect to cause fragmentation of said chains of nucleic acids in the sample in order to form a treated sample; and

subjecting the treated sample to a nucleic acid sequencing operation.

In a thirteenth preferred aspect, the present invention provides a sequencing apparatus, comprising:

a pre-sequencing station, adapted to receive a device according to any one of the sixth to eleventh aspects, for fragmenting chains of nucleic acids in a sample to form a treated sample;

a transfer mechanism;

a sequencing station;

wherein the transfer mechanism connects the pre-sequencing station and the sequencing station and is operable to transfer the treated sample from the pre-sequencing station to the sequencing station, the sequencing station being operable to receive the treated sample and carry out a sequencing operation on the treated sample.

The treated sample may be subjected to sequencing either at the sample treatment zone or the treatment sample may be transferred from the sample treatment zone for sequencing. The transfer of the sample may be carried out for example by pipetting (typically achieved using robotics) or by a microfluidics process (such as a pressure-activated process or electrowetting on dielectric (EWOD) process).

The first, second, third, fourth, fifth, sixth, seventh, eighth, ninth, tenth, eleventh, twelfth and/or thirteenth aspects of the invention may have any one or, to the extent that they are compatible, any combination of the optional features set out with respect to any aspect.

Further optional features of the invention are set out below.

### BRIEF DESCRIPTION OF THE DRAWINGS

Embodiments of the invention will now be described by way of example with reference to the accompanying drawings in which:

FIG. 1 shows a schematic cross sectional view of an embodiment of the present invention in operation.

FIGS. 2, 3 and 4 show electrographs indicating the change in fragment length of DNA subjected to SAWs under different conditions using a device as shown in FIG. 1. For each electrograph, 9 μL of sample containing Genomic DNA (Promega G3041) at a concentration of 25 ng/μL was exposed to 4.86 MHz ultrasonic surface acoustic wave radiation. For FIG. 2 the sample was liquid and 2 W transmitted power was applied for 90 s (temperature less than or equal to 4° C.). For FIG. 3 the sample was liquid but a higher power of 5 W transmitted power was applied for 40 s (temperature less than or equal to 8° C.), the shorter time due to nebulisation of sample—note the appearance of fragments peaking at 1292 bp. For FIG. 4 the sample was partially liquid (i.e. partially frozen) while 2 W of transmitted power was applied for 90 s (temperature less than or equal to 2° C.)—this condition resulting in a desired peak position of sub 1000 bp. Note that time is exponentially linked to size on the x-axis.

FIG. 5 shows a schematic cross sectional view of another embodiment of the present invention in operation, using a superstrate.

FIGS. 6A-6D show different superstrates are shown for use with the arrangement of FIG. 5.

FIGS. 7 and 8 show electrographs indicating the change in fragment length of DNA subjected to SAWs under different conditions using a device as shown in FIG. 5. The electrograph of FIG. 7 was obtained using a flat Si superstrate as in FIG. 6A. The electrograph of FIG. 8 was obtained using a patterned Si superstrate as in FIG. 6D. For each electrograph, 9 μL of sample containing Genomic DNA at a concentration of 25 ng/μL was exposed to 4.86 MHz ultrasonic surface acoustic wave radiation. For FIG. 7 the sample was liquid and 12 W transmitted power was applied for 90 s (temperature less than or equal to 30° C.), with the sample in contact with a flat planar silicon surface. For FIG. 8 the sample was liquid and 12 W transmitted power was applied for 90 s (temperature less than or equal to 30° C.), with the sample in contact with a roughened or patterned planar silicon surface—this condition resulted in a desired peak position of sub 1000 bp.

FIG. 9 shows a schematic cross sectional view of an embodiment of the present invention in operation, in which the superstrate includes an array of cavities.

FIG. 10 shows a schematic cross sectional view of a modified embodiment compared with FIG. 9, in which the cavities include additional projections.

FIG. 11 shows a schematic cross sectional view of another embodiment of the invention, in which the liquid sample is held between the transducer and a superstrate.

FIG. 12 shows a schematic cross sectional view of another embodiment of the invention, in which the liquid sample is held between two transducers.

FIG. 13 shows another embodiment of the invention in which the sample is held in an enclosed chamber at the sample treatment zone.

FIG. 14 shows a modification of the embodiment of FIG. 13.

FIG. 15 shows a further modification of the embodiment of FIG. 13.

FIG. 16 shows another modification of the embodiment of FIG. 13.

FIGS. 17-20 each show an electrograph of samples treated under various different conditions of power, duty cycle and temperature using the embodiment of FIG. 16.

FIG. 21 shows another embodiment of the invention in which the sample is held in an enclosed chamber at the sample treatment zone, the sample being treated using bulk acoustic waves.

FIG. 22 shows a modification of the embodiment of FIG. 21.

FIG. 23 shows a further modification of the embodiment of FIG. 21.

FIG. 24 shows another modification of the embodiment of FIG. 21.

FIG. 25 shows another embodiment of the invention in which the sample is held in an enclosed chamber at the sample treatment zone, the sample being treated using bulk acoustic waves.

FIG. 26 shows a modification of the embodiment of FIG. 25.

FIG. 27 shows a further modification of the embodiment of FIG. 25.

FIG. 28 shows another of the embodiment of FIG. 25.

FIG. 29 shows a part of a sample treatment zone for use in a further embodiment of the invention in which the acoustic wave is a Bleustein-Gulyaev wave.

FIG. 30 shows a part of a sample treatment zone for use in a further embodiment of the invention in which the acoustic wave is a guided Love wave.

FIGS. 31-33 show SEM images of arrays of pits formed at the sample treatment zone of a SAW superstrate.

FIG. 34 shows a mode for using the superstrate of FIGS. 31-33.

FIG. 35 shows DNA fragment distributions for the superstrate of FIGS. 31-33 at different applied powers.

FIGS. 36-38 show SEM images of arrays of pillars formed at the sample treatment zone of a SAW superstrate.

FIG. 39 shows a mode for using the superstrate of FIGS. 36-38.

FIG. 40 shows DNA fragment distributions for the superstrate of FIGS. 36-38 at different applied powers.

FIG. 41 shows an SEM image of an array of pillars formed at the sample treatment zone of a SAW superstrate.

FIG. 42 shows DNA fragment distributions for the superstrate of FIG. 41 at different applied powers.

FIG. 43 shows a mode for using a further superstrate.

FIG. 44 shows DNA fragment distributions for a flat superstrate used as shown in FIG. 43 at different DNA sample concentrations.

FIGS. 45-47 show SEM images of different parts of a roughened Si superstrate.

FIG. 48 shows DNA fragment distributions for a roughened superstrate used as shown in FIG. 43 at different DNA sample concentrations.

FIGS. 49 and 50 show SEM images for pits formed in SU8 subjected to different processing conditions.

FIG. 51 shows an SEM image for a pillar of SU8.

FIG. 52 shows DNA fragment distribution for the superstrate of FIG. 51.

FIG. 53 shows an SEM image for a different pillar of SU8.

FIG. 54 shows DNA fragment distribution for the superstrate of FIG. 53.

FIGS. 55-57 show SEM images for an array of pillars formed of SU8.

FIG. 58 shows DNA fragment distributions for the superstrate of FIGS. 55-57 at different applied powers.

FIGS. 59-61 show SEM images for a trough formed in SU8.

FIG. 62 shows DNA fragment distributions for the superstrate of FIGS. 59-61 at different applied powers.

FIGS. 63-65 show SEM images for a strip formed in SU8.

FIG. 66 compares DNA fragment distributions for the superstrates formed using troughs and strips of different depths.

FIG. 67 compares DNA fragment distributions for the superstrates formed using troughs and strips subjected to different processing conditions.

FIG. 68 shows a flow chart outlining a DNA sequencing process, including a fragmentation step according to an embodiment of the invention.

FIG. 69 shows a plan view of the processing of an interdigitated electrode structure to form a freeze-thaw DNA fragmentation device.

FIG. 70 shows a perspective view of a sample droplet located at the sample treatment zone of a freeze-thaw DNA fragmentation device.

FIG. 71 shows a plot of temperature with position across a freeze-thaw DNA fragmentation device during heating.

FIG. 72 shows a frequency scan of the freeze-thaw DNA fragmentation device using an Agilent vector network analyser (S11 parameter). Marked on the scan is a small trough indicative of small resonance around 32 MHz.

FIG. 73 shows a screenshot from a Bruker Contour GT white light profilometer scan of the surface of the freeze-thaw DNA fragmentation device.

FIG. 74 shows the data of FIG. 73 in plan view.

FIG. 75 shows a plot generated by a Polytec GmbH single point vibrometer (range up to 24 MHz) showing the presence of the first sub harmonic due to the restricted range of the vibrometer used (up to 24 MHz) when excited by a 5V pkpk signal at 32 MHz, indicating some actuation of the surface.

FIG. 76 shows an electrograph of 9 μL of Human DNA (Coriell NA12878) with a concentration of 38 ng\μL placed directly onto the freeze-thaw DNA fragmentation device.

FIG. 77 shows an electrograph of 9 μL of Human DNA (Coriell NA12878) with a concentration of 38 ng\μL placed onto a smooth glass superstrate on the freeze-thaw DNA fragmentation device.

FIG. 78 shows an electrograph of 6 μL of Genomic DNA (Promega G3041) with a concentration of 43 ng/μL placed onto a smooth glass superstrate on a micro strip heater.

FIG. 79 shows an electrograph of 6 μL of Genomic DNA (Promega G3041) with a concentration of 43 ng/μL placed onto a structured silicon superstrate (pegs 130 μm dia. 160 μm high with a pitch of 230 μm) on a micro strip heater.

### DETAILED DESCRIPTION OF THE PREFERRED EMBODIMENTS, AND FURTHER OPTIONAL FEATURES OF THE INVENTION

In the preferred embodiments of the present invention, DNA such as genomic DNA is subject to treatment using SAWs in order to generate DNA fragments of length particularly suitable for automated sequencing. The use of SAWs allows the use of lower sample volumes and lower powers. The typical size and configuration of SAW transducers also enables the integration of fragmentation into sequencing instrumentation. This enables the implementation of sample preparation pre-sequencing steps within the next generation of sequencing instruments. This allows sequencing to be carried out in one integrated instrument, rather than having a stand-alone fragmenting instrument and a stand-alone sequencer, with a skilled operator required to transfer the DNA fragment sample to the sequencer (as is currently the case). This results in reducing total costs for sequencing, increased automation leading to increased throughput, and a broader uptake of the technique across existing and new sectors. The disclosed approach to DNA fragmentation also enables field-based DNA sequencing—as may be required for determining “microbial resistance” and informing the treatment of infectious disease in the face of the emergence of drug resistance (as seen in rare variants of HIV not identified by traditional genotyping techniques.

As will be discussed, the approaches disclosed herein allow the use of a planar geometry, which is of particular interest for the development of a cartridge-based approach to fragmentation. The cartridge can be formed, in part, using the transducer, but more preferably, the cartridge may provide the superstrate used in preferred embodiments of the invention, for coupling with a transducer which forms part of a fragmentation apparatus. In this case, it is preferred that the cartridge is disposable.

In the preferred embodiments of the invention, a liquid sample is placed onto a treatment zone of a SAW transmission surface. The SAW transmission surface supports SAWs in the form of harmonic surface displacements with a frequency of at least 100 kHz, preferably about 1 MHz, and at most 1 GHz or at most 100 MHz. In the most preferred embodiments, the SAW frequency used is in the range 4-10 MHz. SAWs such as Rayleigh waves exist on a solid half space and they exhibit the property of no dispersion. However, other SAWs such as Lamb type waves can be used. Lamb waves are dispersive and this property can be exploited to enhance the amplitude of the surface displacements. The transversal component of these vibrations couple to the liquid sample and radiate compressional waves into the liquid. Due to the difference between the speed of sound in the solid and that of the sample, the compression waves are radiated at an angle which obeys a Snellius type law of refraction. Where there is a free surface of the sample, the longitudinal pressure waves are trapped in the sample due to the acoustic impedance mismatch between air and the liquid and between the liquid and the SAW transmission surface. This is described in more detail in WO 2011/060369, WO 2012/114076, WO 2012/156755 and PCT/GB2014/052672 (WO 2015/033139), the contents of which are hereby incorporated by reference.

The liquid sample shapes the propagation of the sound energy as the air/water interface is a very good reflector of sound as the acoustic impedance mismatch means that 99.99% of the sound wave gets reflected at the interface. This strong reflection at the air/water interface also creates high pressure waves in the sample. Further to this, because a fluid can change shape in response to acoustic forcing, the pressure wave distribution can vary with time. This variation causes differential flows and enhances the shearing of chains of nucleic acids.

It is preferred that the liquid sample is cooled in order to suppress loss of material due to nebulization, if the system is an open system. Furthermore, the sample can be frozen prior to fragmentation using surface acoustic waves. A particularly suitable configuration uses a Peltier cooler, in order to maintain the planarity of the system. In the case where the sample includes a two phase system, the rheology of the two phase system may change with temperature, allowing emulsification to occur and/or increasing the miscibility of the two phases, which may be disadvantageous. Therefore, even in a closed system, cooling can still provide a useful additional effect.

The liquid sample is preferably aqueous. Water can dissolve quantities of gas. This property can be used in order to cause cavitation in the sample due the acoustic strain developed in the liquid. Such acoustic strain can enable the nucleation of bubbles which can then act as cavitation centres. The cavitation centres irradiated by harmonic pressure waves in the medium can be used to further assist in the fragmentation of the chains of nucleic acids. Suitable cavitation centres include surface roughness features. For example, an ordered or non-ordered array of cavities may be produced in the sample treatment zone using deep reactive ion etching, e.g. using the Bosch process. Such an etching process tends to form a scalloped surface to the sidewalls of the cavities, having a nanometre length scale. Such features are suitable nucleation centres. Additionally, the edge/corner where the sidewall meets the base of the cavity provide suitable nucleation centres.

The liquid sample may be treated in order to dissolve gas into it. The gas may be any suitable gas that dissolves in the liquid and which promotes bubble formation. The liquid may be saturated with the gas, or supersaturated with the gas.

The devices according to preferred embodiments of the invention use at least one interdigitated transducer (IDT) fabricated on a piezoelectric substrate to generate SAWs such as Rayleigh-Lamb type elastic waves. The SAWs propagate along a SAW transmission surface to a sample treatment zone to couple into the liquid sample. A compressional wave is radiated into the liquid, because the speed of sound in the liquid is slower than at the SAW transmission surface and the compressional wave is refracted at an angle relative to the normal from the SAW transmission surface. The long chain molecules in the sample interact with the compressional waves propagating in the liquid sample and absorb the mechanical energy. This heats the sample. To control the internal heating of the sample, and thus to control unwanted thermal damage to the DNA, an active cooling device with heat sink is used to extract excess heat from the irradiated liquid. Indeed such temperature control can be achieved by using a pulsed mode of operation where high peak powers are used over a short time period and the rest of the duty cycle is used to allow the sample to cool down.

The acoustic impedance mismatch between air and water is 99.99% implying that almost all the sound wave that impinges on the liquid/air interface is reflected or trapped. This fact allows for energy to be pumped into the liquid and to create pressure at a greater efficiency in the liquid.

The device may form all or part of a chamber that itself forms part of a cartridge or part of a larger microfluidic device. This is discussed in more detail below.

A harmonic signal is applied to the transducer, of frequency typically not less than 1 MHz and not greater than 1 GHz. This generates a harmonic surface displacement, this surface displacement will generate accelerations of the order 10⁶ ms⁻² or higher at the surface of the substrate dependent on the frequency used. Where multiple transducers are used, corresponding signals are applied to the transducers. The magnitude, shape and position of the resultant surface displacements can be controlled by corresponding control of the configuration, frequency, phase and number of transducers.

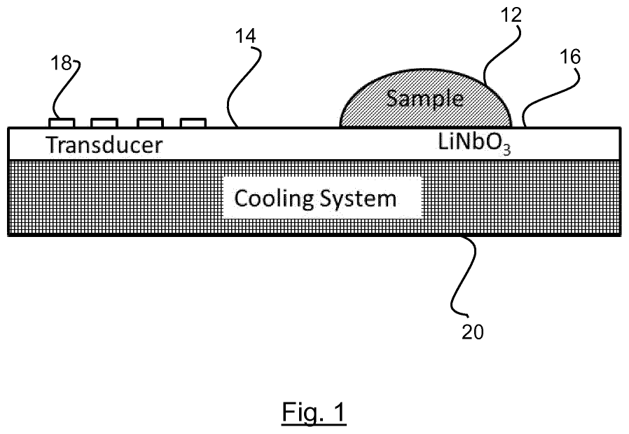

Turning now to an explanation of the embodiments shown in the drawings, it is believed that micro flows generated via acoustic streaming in liquid samples containing DNA or other such polymeric or long chain materials are drivers for fragmentation of the long chain material into smaller parts. In the simplest embodiment of the present invention, shown in FIG. 1, an open geometry is used, in which the liquid sample 12 is placed on a sample treatment zone 16 of a LiNbO₃ SAW transducer 14. SAWs generated by the transducer electrodes 18 are transmitted along the SAW transmission surface to the sample treatment zone 16. The drawing is schematic in the sense that the sample droplet appears large. In practice, the sample droplet is much thinner in height, increasing A/V compared with the impression given by FIG. 1.

One of the drawbacks to using an open geometry for the fragmentation of chains of nucleic acids is the propensity of the sample to nebulise and with it loss of material from the system. This can be overcome to some extent by cooling the sample liquid prior to and subsequent to irradiation by surface acoustic waves. For this reason, as shown in FIG. 1, a cooling system 20 is provided in contact with the lower face of the transducer, in order to extract heat from the transducer and therefore also from the liquid sample.

Acoustic streaming is a second order effect caused by the propagation or the presence s of acoustic vibrations interacting with a fluid. The streaming can induce rapid counter propagating flows in the sample fluid which can tear apart the strands of DNA or other long chain structures of interest. Bubbles can be a localised source for acoustic streaming.

In one approach of an embodiment of the invention, the inventors take advantage of the thermodynamic properties of water where the sample is in a partially frozen state. This allows fragmentation to occur at lower powers than otherwise, and a possible mechanism for this is explained below.

In a specific example of the embodiment schematically shown in FIG. 1, the transducer was based on 1 mm thick Y cut black LiNbO₃ with an electrode spacing to provide a working frequency of 4.86 MHz. The IDT had a dimension of 23 mm square. A Peltier cooler was attached to a fan assisted heatsink using heat sink compound with the transducer attached to the Peltier cooler using the same heat sink compound. This enabled the system to operate in normal ambient temperatures.

In a straightforward modification, the upper surface of the transducer except for the sample treatment zone was treated such that it became hydrophobic. This makes it easier to recover the sample or to process the sample further directly on the device.

With reference to FIG. 1, an aqueous liquid sample 12 of volume about 9 μL was placed as a drop onto the sample treatment zone 16, located spaced apart from the electrodes 18. The liquid sample contained between 25 ng/μL to 100 ng/μL of genomic DNA. During operation, the Peltier cooling device was operated in order to control the temperature of the sample during irradiation with the SAWs generated by the IDT. In this work, the Peltier cooler was operated so that the temperature of the sample did not exceed 37° C.

The surface temperatures of the samples were measured with the aid of a Fluke Ti25 IR camera. The results of the fragmentation were analysed using an Agilent Bioanalyser 2100 with the 12 k kit.

In order to further control the temperature of the liquid sample, the user can pulse the SAW excitation of the liquid sample. By altering the duty cycle, the ratio of time spent on to the time spent off, the average power can be kept low but the peak powers can be allowed to become high without heating of the sample being a problem. However, this requires that the total time to be extended so that the sample sees the required amount of SAW irradiation. For example a duty cycle of 50% on and 50% off will require double the time of a continuously irradiated sample. Typical times used for a continuously irradiated sample were 30 s to 120 s whereas a pulsed 50:50 irradiated sample required at least 60 s to 240 s.

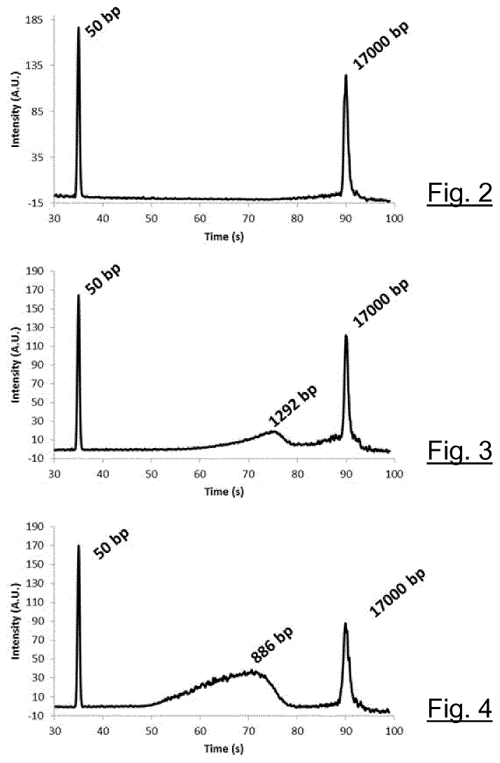

FIGS. 2, 3 and 4 show electrographs carried out on samples of volume 9 μL containing 25 ng/μL of Genomic DNA (Promega G3041). The samples were subjected to 4.86 MHz SAW radiation using the configuration of FIG. 1. For FIG. 2, the sample was liquid and 2 W transmitted power was applied for 90 s. The temperature was less than or equal to 4° C. For FIG. 3, the sample was liquid but a higher power of 5 W transmitted power was applied for 40 s. The temperature was less than or equal to 8° C. A shorter time was used compared with FIG. 2 due to nebulisation of sample. In FIG. 3, note the appearance of fragments peaking at 1292 bp. For FIG. 4, the sample was partially liquid and partially solid while 2 W of transmitted power was applied for 90 s temperature less than or equal to 2° C., this condition resulted in a desired peak position of sub 1000 bp. Note that time is exponentially linked to size on the x-axis.

The power and the amount of time for which the sample is subjected to SAWs has some influence on the position and shape of the fragmented material's distribution. At low power, fragmentation is not apparent. Only when a threshold power is achieved is suitable fragmentation observed. To illustrate this, consider FIGS. 2-4. FIG. 2 uses 2 W and shows no fragmentation (the temperature used for FIG. 2 was less than or equal to 4° C.). FIG. 3 uses 5 W and shows fragmentation (the temperature used for FIG. 3 was less than or equal to 8° C.). Once fragmentation is achieved, the position of the fragmentation peak is substantially insensitive to the power used and will typically remain at approximately 1200 bp (see FIGS. 2 and 3) until very high peak powers are used (>30 W). However, the duration of exposure can have an influence on the shape of the size distribution. With a short duration (e.g. less than 60 s) there are seen distributions with a symmetrical shape (FIG. 3). This morphs into a wider distribution for samples exposed for more than 90 s, even when the temperature is not so high (FIG. 4, in which the temperature was less than or equal to 2° C.). In sequencing applications, a tight size distribution is preferred.

The liquid can be frozen or super cooled prior irradiation with ultrasonic surface acoustic waves. The liquid can be cooled such that only partial melting of the frozen drop is achieved on its surface, when subjected to the ultrasonic actuation. An hypothesis as to the mechanism linked to this could be drawn from results discussed below on the use of superstrates, in that the partially melted liquid is subjected to a roughened interface with the frozen parts. Under such conditions, low powers such as 2 W can be used to achieve the desired fragmentation of below 1000 bp. In this context, the term “liquid sample” is to be understood to include samples which are liquid at room temperature but which may be solidified, e.g. by freezing, and which at least partially liquefy during the process of fragmentation.

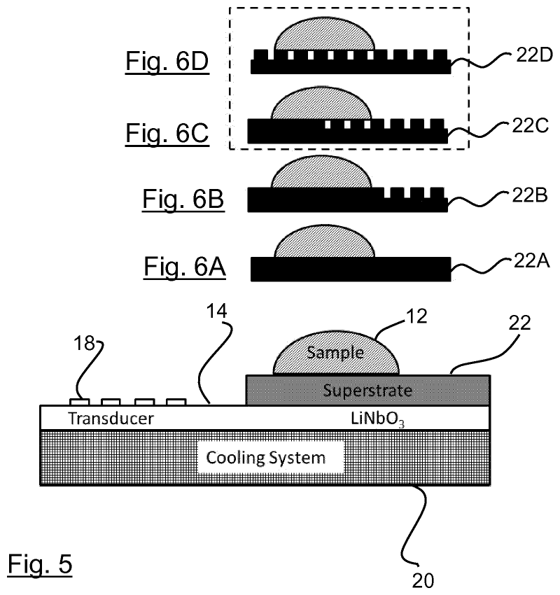

Another embodiment of the invention is illustrated in FIG. 5, which is a modification of the embodiment of FIG. 1. Here, a superstrate 22 is coupled to a transducer 14 such that the mechanical wave can propagate from the transducer to the superstrate 22. In this case, it is the superstrate 22 which provides the SAW transmission surface of interest. The superstrate also provides the sample treatment zone. Parts of the superstrate (other than the sample treatment zone) can be treated to make then hydrophobic, in order to aid to collection of exposed sample. In the examples based on FIG. 5, the sample was cooled prior to treatment using SAWs. During the application of SAWs, the sample melts if frozen, heats up and spreads over the sample treatment zone surface. Depending on the volume of sample, the liquid can spread to form a thin film over the surface. Such a thin film is liable to nebulise and this is to be avoided as there will be loss of material if this is allowed to occur. Ways to avoid nebulisation are to use lower powers, larger volumes or control the temperature of the liquid sample during the fragmentation process by pulsing the excitation. A further way to avoid nebulisation is to use an enclosed sample chamber, which is discussed in more detail below.

FIG. 5 shows a superstrate 22 in direct contact with a surface acoustic wave transducer. Different variations for the superstrate are shown in FIGS. 6A-6D. FIG. 6A shows a planar superstrate 22A which is smooth and flat. FIG. 6B shows a roughened or patterned superstrate 22B where the sample is in contact with a smooth planar part of the superstrate. FIG. 6C shows a roughened or patterned superstrate 22C where the sample is in partial contact with the roughened or patterned part of the superstrate. FIG. 6D shows a roughened or patterned superstrate 22D where the sample is in contact with the roughened or patterned superstrate only. At the time of writing, it is considered that variations used in FIGS. 6C and 6D are particularly suitable.

At different operating frequencies, varying behaviour of the liquid sample can be observed. At lower frequencies, there is significant movement of the drop while at higher operating frequencies the drop can be made to vortex with less translational motion and a similar rate of heating in the liquid sample. This movement can be controlled by modifying the surface chemically by changing the surface chemistry locally or physically by introducing a surface topology which augments the planar surface. The present inventors have used a periodic arrays of pits. In one example a hexagonal array was used with pit diameter 70 μm, depth 50 μm and centre-to-centre spacing 200 μm. In another example a hexagonal array was used with pit diameter 140 μm, depth 70 μm and centre-to-centre spacing 250 μm. The inventors observe that they could have used a random array of such pits or even a mechanically roughened surface formed using abrasive techniques. It is considered that one effect of the surface treatment is that the contact line of the liquid is pinned such that enough power can be applied to fragment the material of interest while having control where the liquid goes. However, the liquid can be allowed to move to the far edge of the superstrate, distal from the electrodes where it will remain throughout the fragmentation process, in effect pinned at the edge.

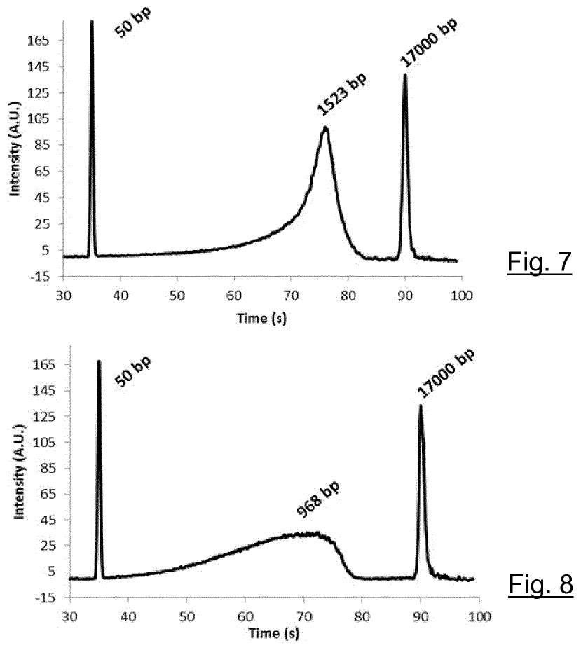

FIGS. 7 and 8 show electrographs of 9 μL of fragmented genomic DNA samples with a concentration of 25 ng/μL exposed to 4.86 MHz ultrasonic surface acoustic wave radiation. For FIG. 7, the sample was liquid and 12 W transmitted power was applied for 90 s. The temperature was less than or equal to 30° C., with the sample in contact with a flat planar silicon surface. For FIG. 8, the sample was liquid and 12 W transmitted power was applied for 90 s. The temperature was less than or equal to 30° C., with the sample in contact with a roughened or patterned planar silicon surface. This arrangement resulted in a desired peak position of less than 1000 bp.

Another advantage of a roughened surface is that it increases the efficiency of the fragmentation process, achieving a desired distribution peak value less than 1000 bp at relatively low applied powers (12 W instead of >30 W). This has the advantage of controlling the temperature of the fragmentation process to be less than 37° C., preferably less than 20° C., thus avoiding any issues of heat stress for any biological samples. The efficiency is achieved by enabling streaming flows to occur adjacent to or on the pitted surface enabling higher shear to occur than would be present on an otherwise flat planar surface.

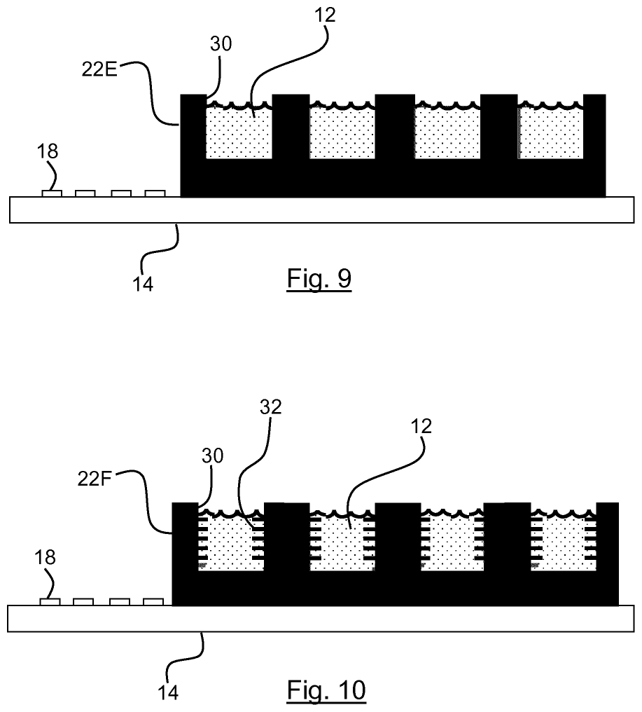

FIGS. 9 and 10 show modified embodiments in which the superstrate is adapted in different ways. The Peltier cooler is not shown, but can be incorporated as described above.

In FIG. 9, an array of cavities 30 is provided in superstrate 22E which hold the sample 12. In this embodiment, the level of the free surface of the sample is below the top of the cavities 30, but it is possible instead for the sample to overfill the cavities. In operation, the cavities are pumped with acoustic energy to amplify the pressures and streaming flows in the sample. Instead of cavities, the superstrate can employ an arrangement of pillars where the sample is free to flow around. The pillars act as scatter sites and as such can create areas of enhanced pressure gradients and hence streaming flows. These structured features of the sample treatment zone are considered to act as sites for the promotion of bubble nucleation.

As shown in FIG. 10, an array of structures 32 can be included on the side walls of the pillars or cavities 30 in order to induce more streaming flows in the fluid sample. FIG. 10 represents a combination of 2D phononic crystal structures to form a 3D phononic crystal structure. The 3D structure can be used to shape the sound field generated by the coupled SAWs into the structure. This embodiment promotes cavitation within the phononic crystal by increasing the sound amplitude and therefore the acoustic strain.

The superstrate may comprise one or more phononic structures in order to affect the distribution of SAWs at the sample treatment zone. A SAW phononic structure is a structure designed to influence the propagation or distribution of SAWs. These phononic structures may be provided as an array of scattering sites or as one scattering site. Details of different arrangements of phononic structures are set out in WO 2011/060369, WO 2012/114076, WO 2012/156755 and PCT/GB2014052672.

Suitable phononic structures can be incorporated in the device in a number of ways. For example, they may be directly attached to the surface of the transducer. They may be constructed using solid material or using a number of gas bubbles in the fluid sample containing biologically relevant polymeric material. The phononic structures may be formed directly onto a superstrate where the phononic structure can be constructed out of solid material or depending on the nature of the superstrate could constructed out of a number of gas bubbles held in place for example by capillary forces due to surface chemistry or surface geometry. The phononic strucures may be formed inside the transducer or superstrate, for example embedded into the superstrate as layers of material with different density and elastic modulus such as an array of fluidic channels.

A simple embodiment of a phononic structure is a thin layer of metal deposited on the surface of a piezoelectric material. The metal shorts out the electrical component associated with the harmonic mechanical deformation of a traveling wave and this has the effect of slowing down the propagation of the SAW. By slowing down the propagation of the leading edge of a traveling wave there rest of wave bunches up, increasing the amplitude of the displacement in an analogous manner to a tsunami. By using this effect the effectiveness of the fragmentation process can be improved by increasing the surface displacement over a small distance and which may cause faster acoustic streaming flows. These flows can be broken up into adjacent regions from an incoming SAW by simply using narrow strips of metal. Multiple transducers operating independently can be used to cause strong acoustic streaming counter flows within the sample. Further, it is known that the use of metallic patterning of the piezoelectric surface can be used to fabricate other dispersive structures such as lenses, beamsplitters and or prisms.

Such displacement increases can be achieved in other ways. For example, they can be achieved by the addition of material onto the surface of either a transducing substrate or the surface of a superstrate where the speed of sound of the added material is lower than that of substrate or superstrate. Polymers such as SU8, glass and/or aerogels can be considered. Such changes in phase velocity can be achieved by using the dispersive properties of a plate (superstrate) where the phase velocity is dependent on the frequency thickness product. By making the superstrate thicker the phase velocity of a particular mode of propagation can be made to slow down. Note however that the A0 mode is an exception to this rule. When an A0 mode is excited in the superstrate then if the thickness of the superstrate was gradually increased, the mode would propagate at progressively higher velocities until it reached the Rayleigh limit. Another simple way to increase the surface displacement is to position the sample at an edge of another discontinuity that causes a significant reflection.

Suitable phononic structures include phononic crystal (PhnC) structures or grating structures such as pits, holes, troughs, strips or pillars. Here holes, pits and troughs are considered as type 1 and pillars and strips as type 2. Such structures are dispersive and their transmission properties are frequency dependent. Suitable structures can be designed based on the behaviour required, such as reflection where no Bloch-Floquet modes exist or transmission where the frequency chosen to drive a transducer will couple to Bloch-Floquet modes that can exist or propagate in the PhC structure.

Type 1 PhnC structures comprise pits, holes or troughs in the transducer or superstrate for example, or in a layer which comprises part of a multilayer superstrate structure. The pits, holes or troughs can behave as cavities supporting particular modes of vibration on the structure or in a fluid and these can be considered closed cavities. The pressure in the cavity can be higher than that of a surrounding fluid. This can be used to improve the probability that fragmentation will occur. However, if desired the pits, holes or troughs can be arranged so as to create an acoustic cavity to enhance the sound field in a particular area. Thus, the whole phononic crystal array can support (Bloch-Floquet) modes and a cavity can be created from a PhnC and excited with SAW. In this manner, a structure can be designed to operate on different length scales, the holes being excited individually at a high frequency or the whole structure at a lower frequency.

Type 2 PhnC structures are those composed of pillars or strips positioned on a surface. The frequencies used can be chosen such that transduced waves from the transducer can couple to Bloch-Floquet modes of the PhnC structure where the scattering of mechanical waves combine to form high pressure point within the spaces between the pillars or strips. Again structures or frequencies can be chosen such that no Bloch-Floquet modes exist therefore reflecting the sound energy. The structures can be arranged to create a cavity to enhance the sound pressure field in a particular area.

There is an intermediate case of the above two where the PhnC structure is embedded into either a transducer or a superstrate. For example arrays of channels may be embedded into the transducer or superstrate. Pillars may be provided in such channels. Therefore, dispersive elements can be engineered to control the shape of the sound field in a similar manner to lenses, beam splitters and prisms.

One problem with using pillars is the high contact angle to water that the structures present. However, the inventors have shown through experiments that such high contact angles can be overcome by the use of SAWs transmissions on a suitable substrate or superstrate where the sound can couple into the liquid and within a short time the liquid wets the pillars and is subsequently dragged down to the structure via capillary action. The use of ultrasound causes the drop to cast micro-droplets which then change the wetting characteristics of the structure. Once the structure is wetted then the power applied can be increased.

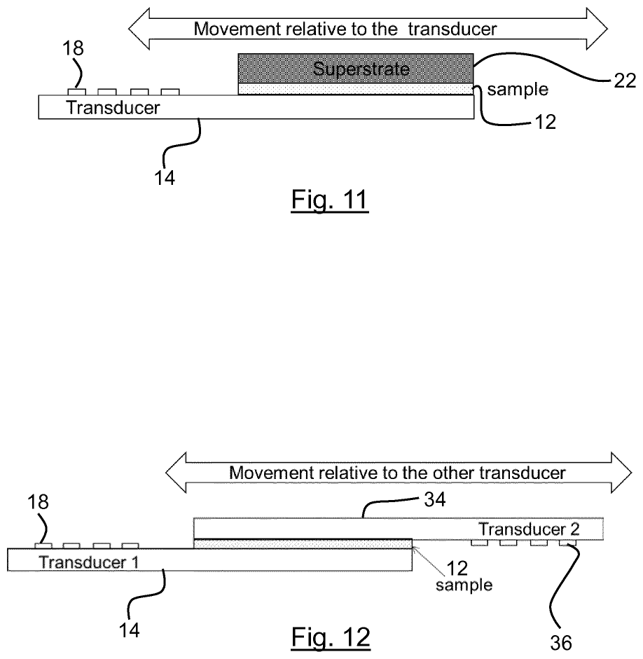

Further embodiments of the invention are now described with reference to FIGS. 11 and 12.

In FIG. 11, a superstrate 22 is provided, but this sandwiches the sample 12 between the transducer 14 and the superstrate 22. The effect of this is that the sample can be more readily contained, and nebulisation reduced or prevented. As shown in FIG. 11, the superstrate 22 can be moved relative to the transducer. Containing the sample in this way allows the ultrasonic waves in the sample (coupled from the SAWs from the transducer) to pass into the superstrate 22. The provision of the additional surface for inducing streaming aids the fragmentation of the chains of nucleic acids. In a modification of this embodiment, the perimeter of the sample may be frozen in order to further limit nebulisation loss from the sample.

In FIG. 12, FIG. 11 is modified so that the superstrate is a transducer superstrate 34, having electrodes 36. This allows the phase, frequency, amplitude and duty cycle of each transducer 14, 34 to be altered in order to further control the fragmentation in the liquid sample 12.

In another embodiment (not illustrated), an intervening superstrate can be inserted between the opposing transducers. In this configuration, the intervening superstrate may be narrower or wider than one or both of the opposing transducers.

In FIG. 11, SAWs couple into the sample 12 and are then transmitted to the superstrate 22 where surface displacements at both the substrate and superstrate interact in order to fragment the chains of nucleic acids. As shown in FIG. 11, the superstrate 22 may be translated relative to the substrate (transducer 14) in order to improve the efficiency.

In a modification of FIG. 11, another embodiment (not illustrated) uses a circular substrate sandwiching the sample between the superstrate and the transducer. The circular substrate can be rotated relative to the transducer. Such rotation has been shown to be effective by Shilton et al (2012) in which SAWs drive rotation of the circular rotor. In effect, the rotor can be considered as a small milling stone that assists in DNA fragmentation by generating differential shear.

The arrangement in FIG. 11 serves to increase the shear to the DNA. Moving the superstrate relative to the substrate ensures that the sample is subjected to a larger range of flow intensities and directions, thus again maximising impact.

In FIG. 12, two IDTs 14, 34 are coupled together by a layer of liquid sample 12. Surface waves couple into the sample and are then transmitted to each transducer and back again where surface displacements at both transducers interact in order to fragment the chains of nucleic acids. The driving frequency of each transducer need not be the same and indeed in some cases it is advantageous if they are not. As in FIG. 11, the transducers may be translated relative to each other in order to improve the efficiency.

As will be clear from the disclosure above, the preferred embodiments of the present invention seek to provide efficient and effective DNA fragmentation at relatively low applied power. Existing literature points towards the fact that micro flows generated via acoustic streaming and/or cavitation in liquid samples containing DNA or other such polymeric or long chain materials are drivers for fragmentation of the long chain material into smaller parts. However, these drivers typically have not been accessible in the case of low ultrasonic radiation powers. The preferred embodiments of the invention enable fragmentation using electrical powers associated with portable hand held devices. This opens up the possibility for the field use of next generation sequencing.

The embodiments of the invention illustrated in FIGS. 1, 5, 6A-6E, 9 and 10 show DNA fragmentation in open systems, i.e. where a drop of liquid sample is manipulated at a surface of the device and the sample presents a free surface. Such embodiments are advantageous for their ease of access, their ease of and implementation and low costs (associated with simple planar geometries). One of the drawbacks to using an open geometry for the fragmentation of polymeric long chain materials such as DNA is the propensity of the sample to nebulise and with it loss of material from the sample. This also leads to partial denaturation, which creates DNA structures that are not useable by sequencing methodologies (e.g. asymmetric, single base pair mis-pairing). This can be overcome to some extent by cooling the sample prior to and during irradiation by surface acoustic waves.

Acoustic streaming is a second order effect caused by the propagation or the presence of acoustic vibrations interacting with a fluid. The streaming can induce rapid counter propagating flows in the sample fluid which can tear apart the strands of DNA or other long chain structures of interest. Also such streaming flows can be induced via cavitation where micro bubbles (of dissolved gas for example) oscillate due to the presence of ultrasonic pressure waves.

Preferred embodiments of the invention take advantage of the thermodynamic properties of water where the sample is in a partially frozen state to allow fragmentation to occur at lower powers than otherwise.

In one suitable embodiment, the invention uses an interdigitated transducer (IDT) on a piezoelectric material such as LiNbO₃ (or any other suitable material that can produce surface vibrations of the desired amplitude and frequency). The results reported here were obtained using 1 mm thick Y cut black LiNbO₃ with an electrode spacing to provide a working frequency of 4.86 MHz, the transducer having a dimension of 23 mm square. A Peltier cooler was attached to a fan assisted heatsink using heat sink compound with the transducer attached to the Peltier cooler using the same heat sink compound. This enabled the system to operate in normal ambient temperatures. The system is as illustrated in FIG. 1. The surface of the transducer can be treated such that it becomes hydrophobic. This makes the recovery of sample, or its further processing directly on the device, easier for a planar geometry.

In the disclosure above, a drop of liquid sample containing between 25 ng/μL to 100 ng/μL DNA onto the surface of the IDT a suitable distance away from the electrodes. In order to control the temperature of the sample during irradiation from the surface acoustic waves generated by the IDT a cooling device is used such as a Peltier type device. The Peltier device was operated so that the temperature of the sample did not exceed 37° C. The surface temperatures of the samples were measured with the aid of a Fluke Ti25 IR camera. The results of the fragmentation were analysed using an Agilent Bioanalyser 2100 with the 12 k DNA kit. The effect of providing active cooling was found to be that nebulisation of the sample was reduced, compared with the same conditions except without active cooling.

A further way to control the temperature of the liquid sample is to pulse the transducer. This has been disclosed also in Yeo et al, Lab Chip. 2014 14(11):1858-65. doi: 10.1039/c4lc00232f. By altering the duty cycle, the ratio of on to off, the average power can be kept low but the peak powers can be allowed to become high without heating of the sample being a problem. However, this requires that the total time to be extended so that the sample sees the same amount of ultrasonic wave irradiation. For example a duty cycle of 50% on and 50% off will require double the time of a continuously irradiated sample. Typical times used for a continuously irradiated sample would be 30 s to 120 s whereas a pulsed 50:50 irradiated sample would require at least 60 s to 240 s. Although not dramatically problematic for sequencing applications, the shorter the time, the higher the throughput of processing, which is one of the major parameters in sequencing applications.

The power and the amount of time taken for the fragmentation have some influence on the position and shape of the fragmented material's distribution. At low powers, fragmentation is not observed and only when a threshold power is used is fragmentation achieved. As mentioned above, FIG. 2 uses 2 W and shows no fragmentation (temperature less than or equal to 4° C.) while FIG. 3 uses 5 W and shows fragmentation (temperature less than or equal to 8° C.). Once fragmentation is achieved, in an open system, the position of the fragmentation peak appears to be insensitive to the power used and will typically remain at approximately 1200 bp (see FIGS. 2 and 3) until very high peak powers are used (>30 W). However, the duration of exposure can have an influence on the shape of the size distribution with short time exposures less than 60 s having a symmetrical shape (FIG. 3) with this morphing into a wider distribution for samples exposed for more than 90 s, as shown in FIG. 4 (temperature less than or equal to 2° C.). In sequencing applications, a tight distribution is preferred.

The liquid was frozen or super cooled prior irradiation with ultrasonic waves, cooled such that only partial melting of the frozen drop was achieved during the irradiation with ultrasonic waves. Under such conditions, low powers can be used to achieve the desired fragmentation of below 1000 bp. It is not known at this time the mechanism by which fragmentation is occurring at these low temperatures and applied powers, where sample surface temperature is below or around 4° C. and applied power can be less than 1 W.

Without wishing to be limited by theory, a possible mechanism for low temperature and power DNA fragmentation could be that the partially melted sample subjects the liquid phase to a roughened interface due to the presence of the frozen parts. Additionally or alternatively the frozen parts may be free to move within the liquid phase to mechanically break up the DNA in the presence of a harmonic forcing cause by the transducer. However, the explanation of the phenomenon might be due to thermodynamic considerations related to repeated cycles of crystallization, thawing and recrystallization or even a combination of these effects.

The article provided at: http://www.mlo-online.com/freeze-thaw-cycles-and-nucleic-acid-stability-whats-safe-for-your-samples.php [accessed 7 Oct. 2016] provides some disclosure on the effect of freeze-thaw cycles on DNA. However, this method is apparently not reliable and does not provide good sizes (there is no relevant data provided). See also: http://online.liebertpub.com/doi/pdf/10.1089/bio.2011.0016 [accessed 7 Oct. 2016]

The embodiments of the invention illustrated in FIGS. 5 and 6A-6E utilise a superstrate coupled to a transducer such that the mechanical wave can propagate from the transducer to the superstrate. The superstrate provides the sample treatment zone. The surface of the superstrate can be treated to make it hydrophobic as an aid to collection of exposed sample. The sample is preferably cooled prior to exposure to ultrasonic surface acoustic waves. The liquid sample will heat up (melt if frozen) and spread over the surface. Depending on the volume of sample used, the fluid can make a thin film over the surface. Such a thin film is liable to nebulise and this is preferably reduced or avoided as there will be loss of material if this is allowed to occur. Suitable approaches for reducing or avoiding nebulisation are to use lower powers, larger volumes or control the temperature of the liquid sample during the fragmentation process by pulsing the excitation.

At different operating frequencies, varying behaviour of the liquid sample can be observed. At lower frequencies there is significant movement of the drop (i.e. motion of the drop shape), while at higher operating frequencies the drop can be made to vortex (motion of the liquid inside the drop) with less translational motion and a similar rate of heating in the liquid sample. This movement can be controlled by modifying the surface chemically by changing the surface chemistry locally or physically by introducing a surface topology which augments the planar surface. We have chosen to use a periodic array of pits (about 186 μm diameter, 203 μm pitch) but we could have used a random array of such pits or even a mechanically roughened surface using abrasive techniques. What is considered to be important is that the liquid's contact line is pinned such that enough power can be applied to fragment the material of interest while having control where the liquid goes. In some embodiments the liquid can be allowed to move to the far edge of the superstrate where it will remain throughout the fragmentation process, in effect pinned at the edge.

The effect of using a roughened surface at the sample treatment zone is illustrated by comparing FIGS. 7 and 8.

The use of a roughened surface at the sample treatment zone appears to have the effect of increasing the efficiency of the fragmentation process, achieving a desired distribution peak value less than 1000 bp at relatively low applied powers (12 W instead of >30 W). This has the advantage of permitting control of the temperature of the fragmentation process to be less than 37° C., preferably less than 20° C., thus avoiding serious issues of heat stress for biological samples. The efficiency is achieved by enabling streaming flows to occur adjacent to or on the roughened (e.g. pitted) surface enabling higher shear to occur than would be present on an otherwise flat planar surface.

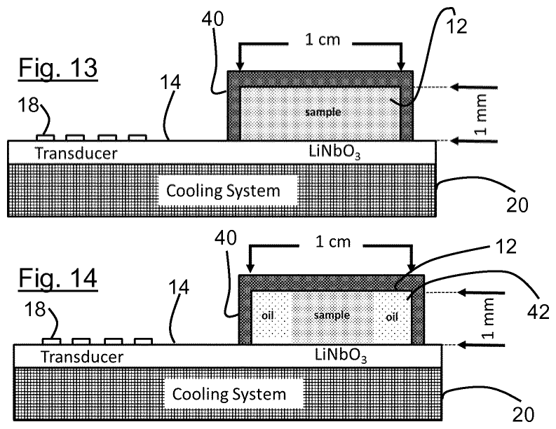

The embodiments discussed above use an open system in which the liquid sample presents a free surface. With power budgets in the region of 1 W, it would be advantageous for the sample to be enclosed in a microfluidic structure. It is preferred for example that the sample is located in a sample chamber 40, 50 (see FIGS. 13-16), in order to enclose it during the fragmentation process while reducing or avoiding sample loss due to nebulisation.

In the simplest approach, the sample chamber 40 may hold a single phase, i.e. the sample 12 (see FIG. 13). Alternatively, the sample chamber 40 may hold a two-phase system where a water based sample 12 is adjacent to and/or surrounded by an immiscible oil phase 42. Such a system will exhibit excessive damping and be prone to emulsification due to the interaction of an intense acoustic irradiation with the immiscible liquids. Indeed this is what we observe for high powers when it is difficult to keep the temperature suitably low as heat generation is a problem. However, it is advantageous that the preferred embodiments of the present invention are compatible with such encapsulation technologies, allowing suitable DNA fragmentation, in order that the preferred embodiments of the invention can be incorporated in existing sequencing workflows, in particular those which use electrowetting on dielectric (EWOD) techniques in order to manipulate the sample.

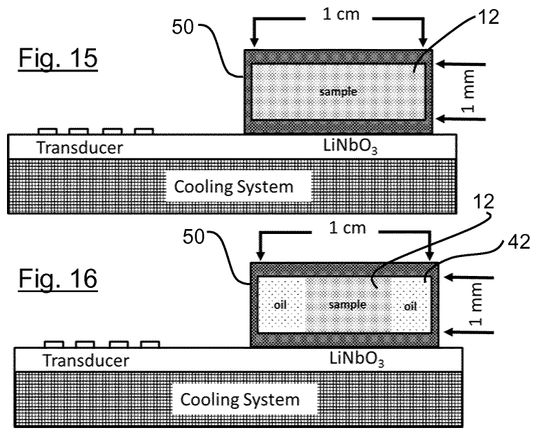

Suitable sample chambers 40, 50 were formed as microfluidic structures fabricated from glass and silicon where the glass was 1 mm thick and the silicon was 500 μm thick. Two approaches were used. The first approach was one where the microfluidic structure was bonded directly to the surface of the transducer with epoxy (FIGS. 13 and 14). The second approach was one where the microfluidic structure comprising a glass top and sides had a silicon base was coupled to the transducer using a KY gel (FIGS. 15 and 16).

In the prior art, it is known to enclose the sample in the chamber where the sample is exposed to high power ultrasonic pressure waves. Part of the insight of the present invention is that where substantial powers are used, there is a need for pulsed irradiation and active cooling such that the temperature of the sample does not exceed 37° C., otherwise the sample will be subjected to temperatures that may denature or damage proteins or DNA.

It is clear from the work reported here that there is a minimum magnitude of ultrasonic excitation required to fragment DNA. However, we have shown that this can be dramatically reduced by choosing suitable conditions, namely control over temperature, for example to ensure that the maximum temperature experienced by the sample is approximately 4° C. We used a number of frequencies ranging from 7.38 to 9.156 MHz and different modes of operation initially pulsed to ensure peak pressures high enough, then continuously when suitable conditions were found. The concentration of DNA used in this work varied from 7 ng/μL to 40 ng/μL, results for 12 and 20 ng/μL being shown here.

In FIGS. 13 and 14 the sample 12 is in direct contact with the piezoelectric surface, but elsewhere contained by the sample chamber 40. In FIGS. 15 and 16 a wall of the sample chamber 50 is interposed between the piezoelectric surface and the sample 12. In effect, FIGS. 13 and 14 correspond to treating the sample directly on the piezoelectric surface and FIGS. 15 and 16 correspond to treating the sample on a superstrate.

Note that in the present work no specific advantage was determined by fragmenting directly on the piezoelectric surface compared with fragmenting on a superstrate. However, it is possible that an effect is seen, and this may offer a route to further reducing acoustic powers required to fragment DNA.

In the embodiments shown in FIGS. 13-16, the depth of the sample chamber 40, 50 was chosen with respect to integration with existing digital microfluidic platforms. However it is envisaged that sample depth is not a critical factor and could be varied without strong dependence on choice of frequency. In FIGS. 14 and 16, the sample 12 is shown in contact with the top and bottom interior surfaces of the chamber, but this is not a fundamental requirement and the liquid can be surrounded by the oil/wax phase. One suitable way to achieve this is to freeze the liquid sample in order to ensure that it can be encapsulated in the oil/wax phase. If the liquids are immiscible, there may be no need to freeze in order to achieve encapsulation.

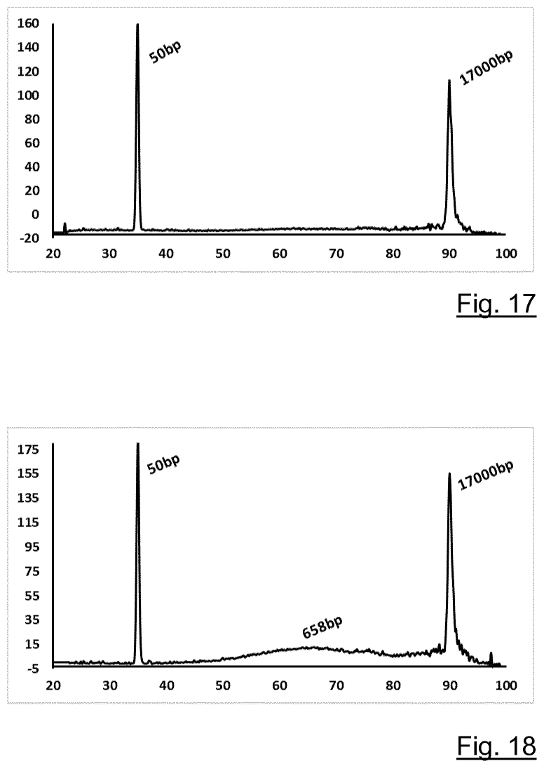

We can process a chamber filled with a single phase namely the sample, however, it is not entirely clear that such a system could handle a two phase system found in digital microfluidics platforms. One concern mentioned above is the preponderance for the two phases to mix and generate an emulsion which would be deleterious to the operation of an electro wetting on demand (EWOD) system. Emulsification was readily observed at elevated temperatures (>10° C.) and high peak powers (36 W corresponding to input of 400 mV pkpk) when using pulsed mode of excitation. This was suppressed when the device was suitably cooled allowing input signals of 600 mV pkpk to be used (corresponding to 290 W peak power). The electrographs shown in FIGS. 17-20 are based on samples treated in a two phase system where the water based sample is surrounded by an immiscible oil. Therefore we have shown that the present invention can work satisfactorily with a two phase system used in digital microfluidics platforms.

FIGS. 17-20 demonstrates the importance of temperature control when fragmenting DNA with relatively low applied average power. In FIG. 17, the SAW frequency was 9.03 MHz with 7 W transmitted power (pulsed 150 k cycles each 200 ms) for 130 s. The liquid sample had a DNA concentration of 12 ng/μL, with the temperature of the sample controlled to be maximum 7° C. In FIG. 18, the SAW frequency was 9.03 MHz with 4 W transmitted power (pulsed 80 k cycles each 200 ms) for 130 s. The sample was partially frozen and had a DNA concentration of 12 ng/μL, with the temperature of the sample controlled to be maximum 1° C.

As can be seen by comparing FIGS. 17 and 18, control of the temperature is important also in this enclosed system, as for the open system discussed above. When the sample temperature was allowed to raise above 7° C., no fragmentation was observed (FIG. 17), compared to FIG. 18. Note that in FIG. 18, the average transmitted power was less than for FIG. 17, and yet a greater degree of fragmentation is seen in FIG. 18.

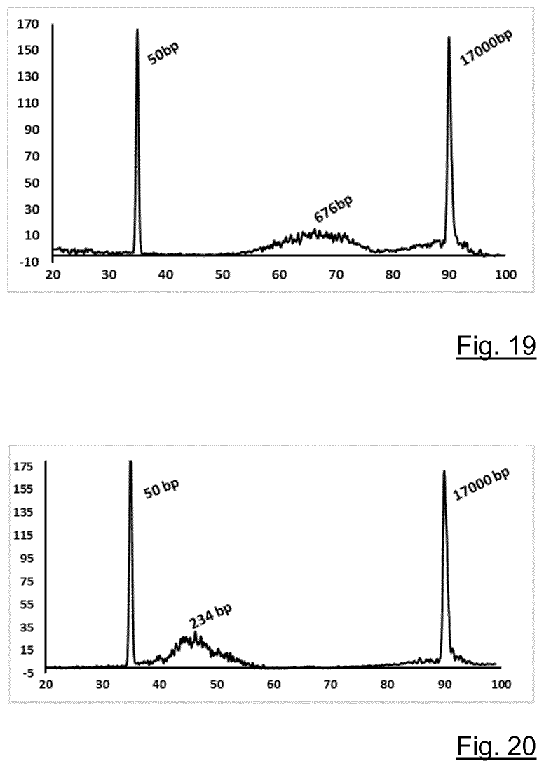

FIGS. 19 and 20 show that suitable control of the treatment conditions permits enough power to couple from a transducer through a superstrate in order to fragment DNA in a device of the configuration shown in FIG. 16. In FIG. 19, the SAW frequency was 9.156 MHz and 2 W transmitted power was used (continuous 110 mV pkpk input signal) for 240 s. The sample was partially frozen with a DNA concentration of 20 ng/μL. In FIG. 20, the SAW frequency was 7.85 MHz and 5 W transmitted power was used (continuous 110 mV pkpk input signal) for 133 s. The sample was partially frozen with a DNA concentration of 20 ng/μL. The maximum temperature of the sample during the process was approximately 2° C. The amount of power coupled into the sample is shown to have a stronger influence on the resultant peak fragment distribution than time of exposure as evidenced by FIG. 20 compared to FIG. 19. Both samples were at approximately the same temperature, showing that the amplitude of the vibration (power applied) has a greater effect than the duration of treatment in order to promote reduced fragment size.

The configuration of FIGS. 15 and 16, where the sample is coupled to but not in direct contact with the transducer can be considered as bulk wave excitation. In this configuration it was noticed that there was more control over the final peak fragment size.

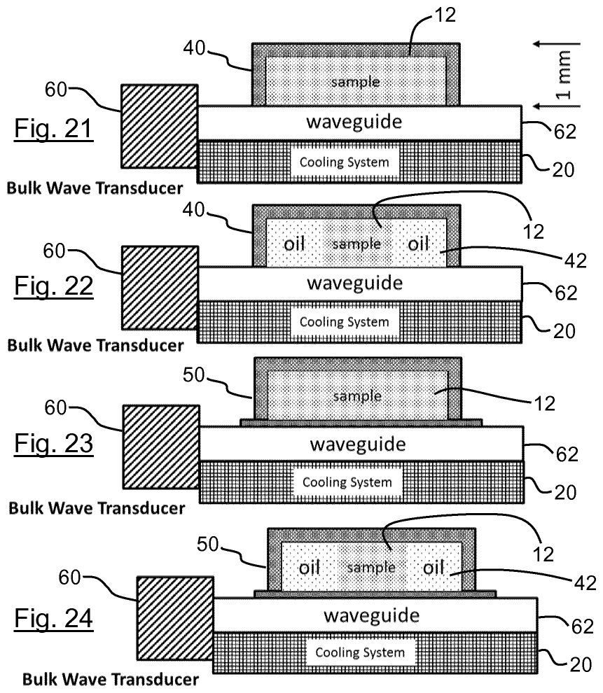

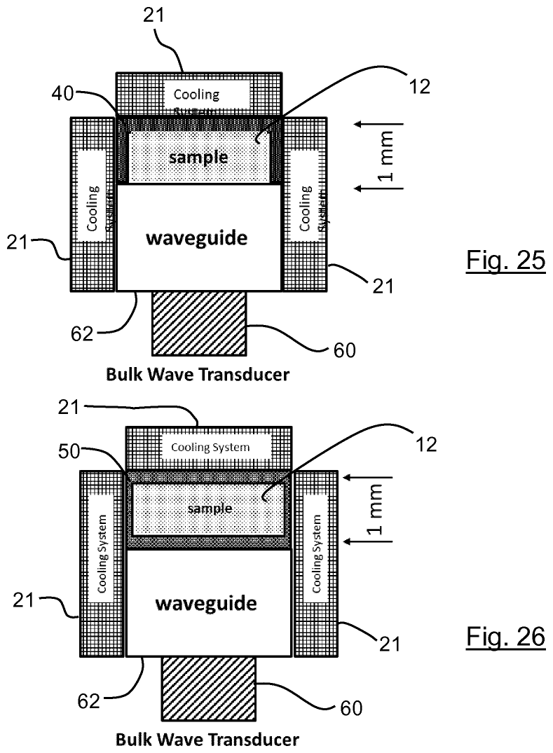

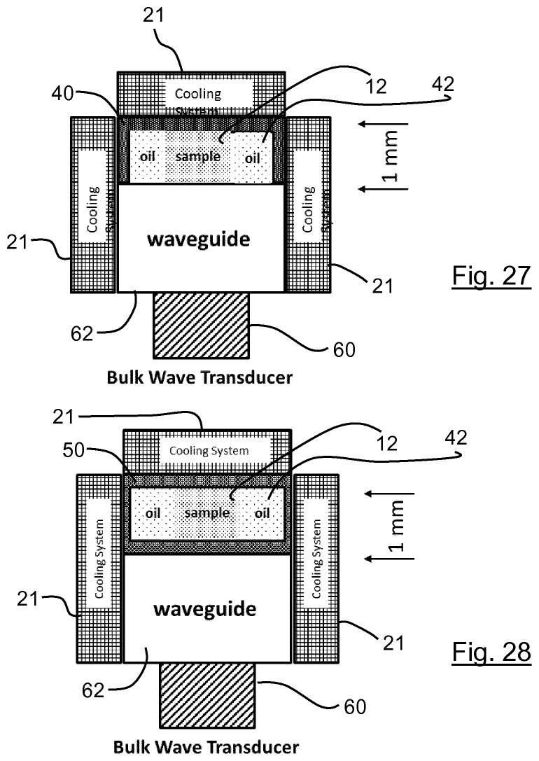

The embodiments illustrated so far use SAW transducers. However, embodiments of the invention also work satisfactorily with bulk acoustic waves, generated using bulk acoustic wave transducers 60 transmitted via waveguide 62. Suitable configurations for such devices are shown in FIGS. 21-24 (these configurations use Langevin type bulk wave transducers 60 to achieve fragmentation of DNA in sample 12) and FIGS. 25-28 (these alternative configurations also use Langevin type bulk wave transducers 60 and waveguides 62 to achieve fragmentation of DNA). In FIGS. 25-28, cooling systems 21 are disposed around the sample chamber 40, 50 holding sample 12 (optionally with immiscible phase 42).

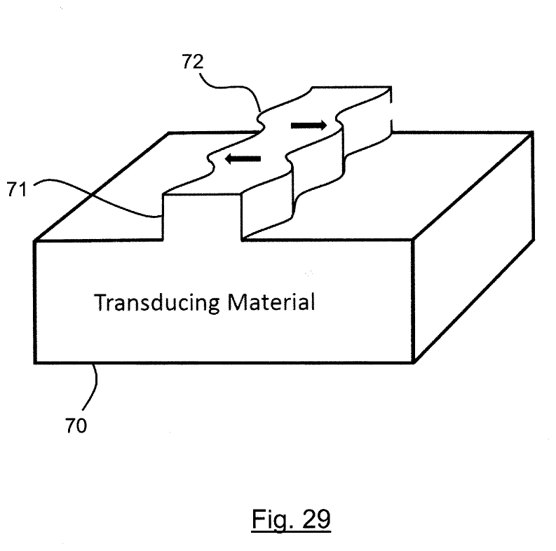

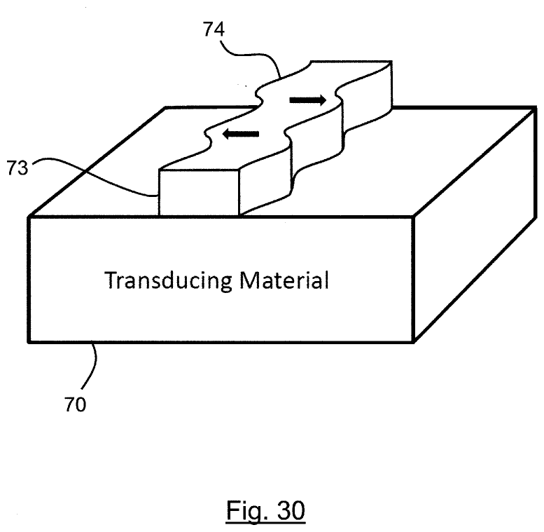

Embodiments of the present invention also work satisfactorily using surface shear waves. This is illustrated using the embodiments shown in FIGS. 29 and 30. FIG. 29 shows a part of a sample treatment zone showing the transducing material 70 in which the acoustic wave is a Bleustein-Gulyaev wave 72, schematically illustrated.

FIG. 30 shows a part of a sample treatment zone showing the transducing material 70 and in which the acoustic wave is a guided Love wave 74, schematically illustrated.

In FIG. 29, the transducer includes a raised ridge 71. As shown, the raised ridge is formed of the piezoelectric material and is formed monolithically with the remainder of the piezoelectric material. Shear waves (Bleustein-Gulyaev) propagate along the transducer, including along the raised ridge 71. At the side walls of the ridge, the effect seen is similar to a Rayleigh or Lamb wave turned on its side. This can couple into the liquid sample as for a Rayleigh or Lamb wave propagating on a planar surface. Additionally, the movement of the side walls provides inertial forcing of the liquid sample and for the promotion of the onset of cavitation.

FIG. 30 shows a similar arrangement to FIG. 29 except that the raised ridge 73 is not formed of a piezoelectric material. Instead, it is formed as a waveguide, forced to oscillate as shown by Love waves, and provides similar effects to FIG. 29 in the liquid sample.

Further work has been completed by the inventors to investigate the effects of the shape of the interface between the sample and the sample treatment zone. This work has demonstrated that the use of an engineered structure at the interface can have a positive influence on DNA fragmentation via ultrasonic waves acting on a sample containing the DNA. This is the case even while the liquid temperature is kept many degrees centigrade above freezing.

In the work reported here, the structures used had varying forms (pit, trough, pillar and strip) and varying edge curvatures, depths and heights. Additionally, the structures were formed of different materials. Some superstrates were completely made out of silicon while others were made out of a patterned layer of SU8 (photresist) on a silicon superstrate.

All the samples were frozen prior to the application of SAW. This was to ensure that all runs had similar starting conditions.

In these experiments the applied power was approximately 5 W to 15 W but generally 13 W was used from an input to the amplifier of 190 mV pkpk. At this power it is found that the temperature of the liquid does not rise too quickly and fragmentation sizes sub 17000 bp can be produced reproducibly.

The LiNbO₃ interdigitated transducer was driven at a frequency of approximately 7.3 MHz and was pasted to a Peltier heater/cooler which in turn was pasted to a heatsink used in conjunction with a 12V fan. Temperature of the liquid samples was measured remotely using a Fluke Ti 25 IR thermal imaging camera. The maximum temperature was 53° C. and the minimum −20° C. although generally the temperature was kept to below 40° C. and above −17° C.

The silicon superstrates were patterned using optical lithography, this photoresist pattern was then transferred into silicon via a dry etch process. The SU8 structures were fabricated using optical lithography. The freeze thaw device was made using metal lift off on a polymeric surface which could be SU8 on silicon or a piece of plastic such as PMMA. With respect to the topologies of the superstrates, the pits or posts were arranged in either triangular or square lattices.

A Vectawave 80 W RF amplifier used in conjunction with RF power meter. The range of temperatures that all samples shown reached during the application of SAW, was between 10° C. and 41° C. A number of various structures were used arrays of pits or posts and flat or roughened silicon. The source of the DNA used came from Corriel (NA12878) at a concentration of 38 ng/μL or 76 ng/μL with 9 μL used for all runs.

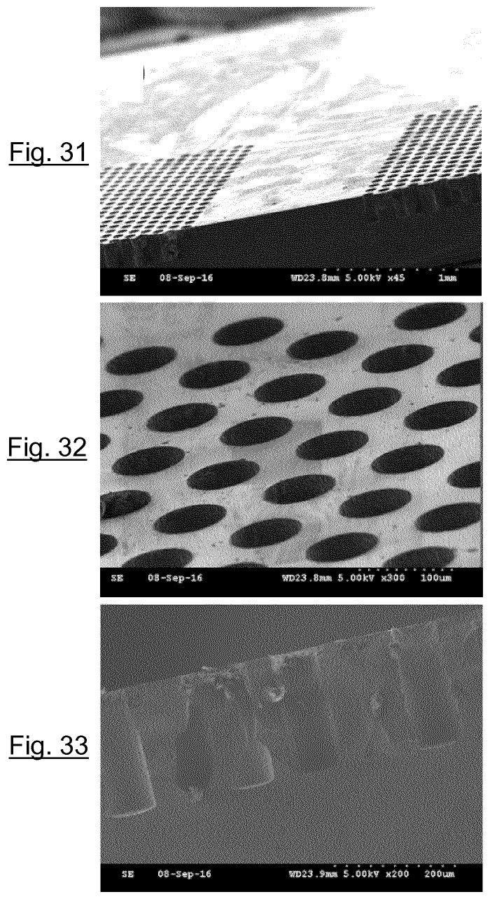

The minimum depth of the Si pits was approximately 80 μm and maximum was approximately 200 μm. The minimum height of the posts was approximately 45 μm and the maximum height was approximately 145 μm. Smallest feature size was 20 μm in diameter and the largest was 1000 μm in diameter. All silicon superstrates were fabricated using (100) oriented 500 μm thick four inch single side polished wafers. An example of the arrayed pit structures is shown in FIGS. 31-33. FIGS. 31 and 32 show perspective SEM views of a silicon superstrate with a triangular lattice of pits, the pits having diameter 75 μm, pitch 120 μm and depth 200 μm. FIG. 33 shows a cross sectional SEM view of the pits.

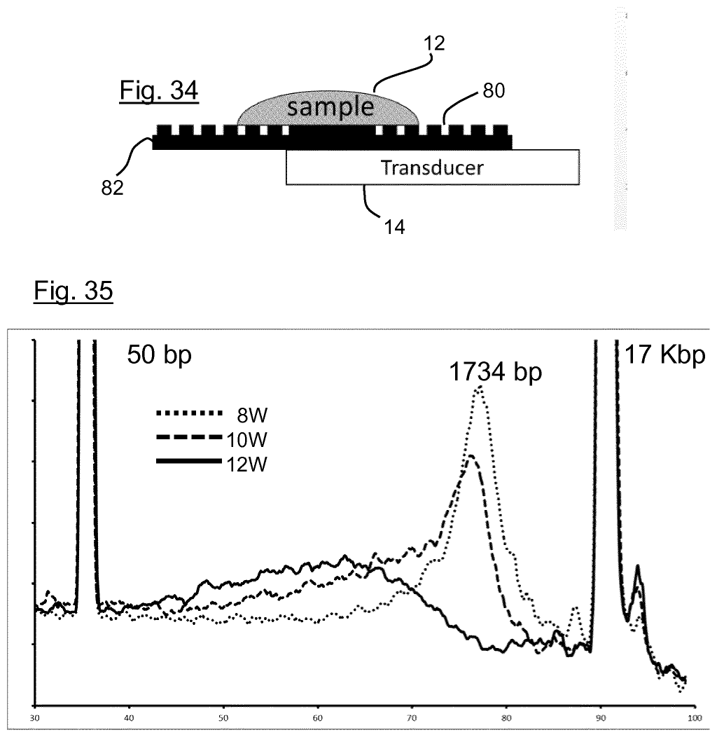

With the embodiment of FIGS. 31-33, the sample was initially placed within a ‘phononic’ cavity (at the flat surface between the two arrays of pits shown in FIG. 31). However, on application of the SAWs, the sample 12 spread to be in direct contact with the pits 80 in the superstrate 82, as shown in FIG. 34.

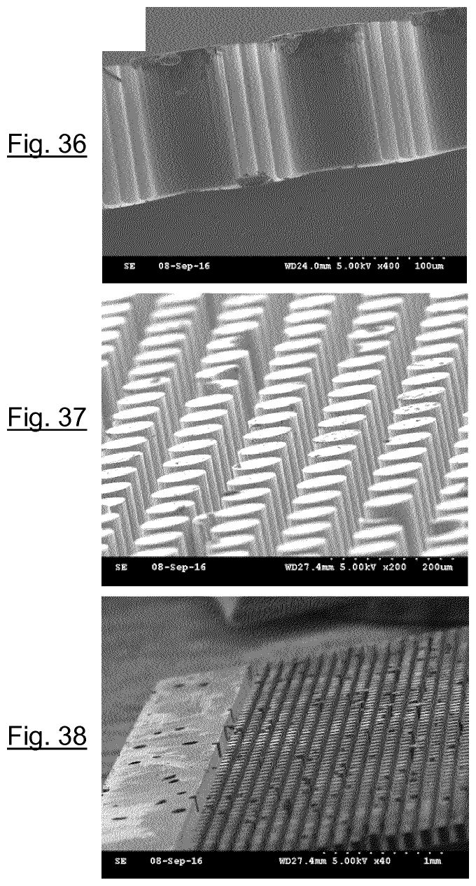

FIG. 35 shows fragment distributions for 9 μL samples for different applied powers using the superstrate of FIGS. 31-33. The peak fragment size was approximately 1734 bp using approximately 7.3 MHz for 120 s. As can be seen, the DNA readily fragments with peak sizes below 2 kbp on the pit structures and above 1700 bp and giving rise to relatively sharp peaks with less than 13 W applied power.

FIGS. 36-38 show SEM images of a silicon superstrate with a square array of pillars. The pillars have diameter 80 μm, pitch 125 μm and depth 144 μm.

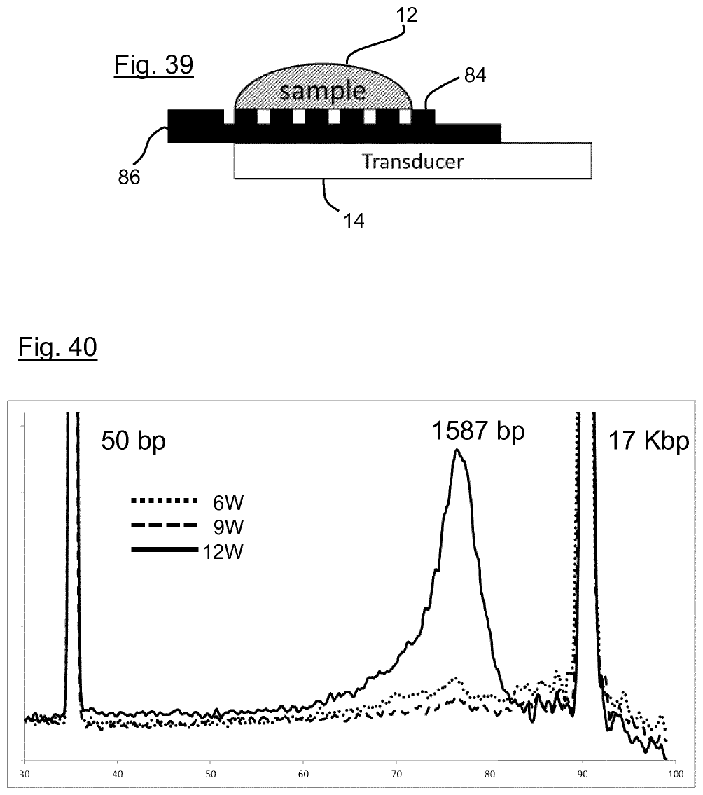

The experimental set up for the superstrate 86 of FIGS. 36-38 is shown in FIG. 39, with the liquid sample placed on the pillars 84. FIG. 40 shows fragment distributions for 9 μL samples for different applied powers. The peak fragment size was approximately 1587 bp using approximately 7.3 MHz for 120 s, note, the yield is higher than for the pit structure superstrate. Thus, it appears that the pillar structures performed better than the pit structures, with all runs producing fragment distribution peaks below 1600 bp and typically above 1100 bp, again giving rise to a relatively sharp peak for applied powers less than 13 W, as shown in FIG. 40.

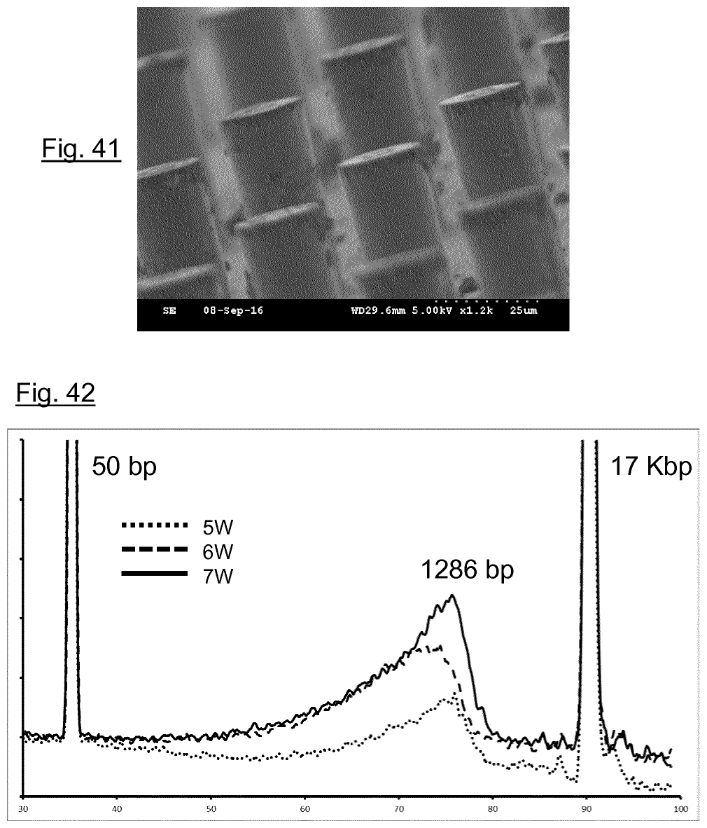

FIG. 41 shows an SEM image of another engineered Si superstrate, with a square array of pillars of height 44 μm, diameter 20 μm and pitch 80 μm. Using a similar arrangement to FIG. 39, a sample was placed on the pillars and subjected to SAWs. FIG. 42 shows the resultant DNA fragment distribution for different applied powers.

Without wishing to be bound by theory, the inventors speculate that the fragmentation yield from the pillar structures is higher than that from the pit structures perhaps because the pillars have a greater degree of freedom to move or provide a larger interaction area with the sample. Also the open structure of a lattice of pillars may promote streaming flows within the liquid sample more effectively that an array of pits.

The roughness of the superstrate surface, apart from the engineered surface structures (pits or pillars) did not appear to be critical to the fragmentation of DNA under the conditions used. However, as expected the intensity of the acoustic field (magnitude of the elastic waves) influences fragment sizes produced with higher powers typically producing smaller fragments.

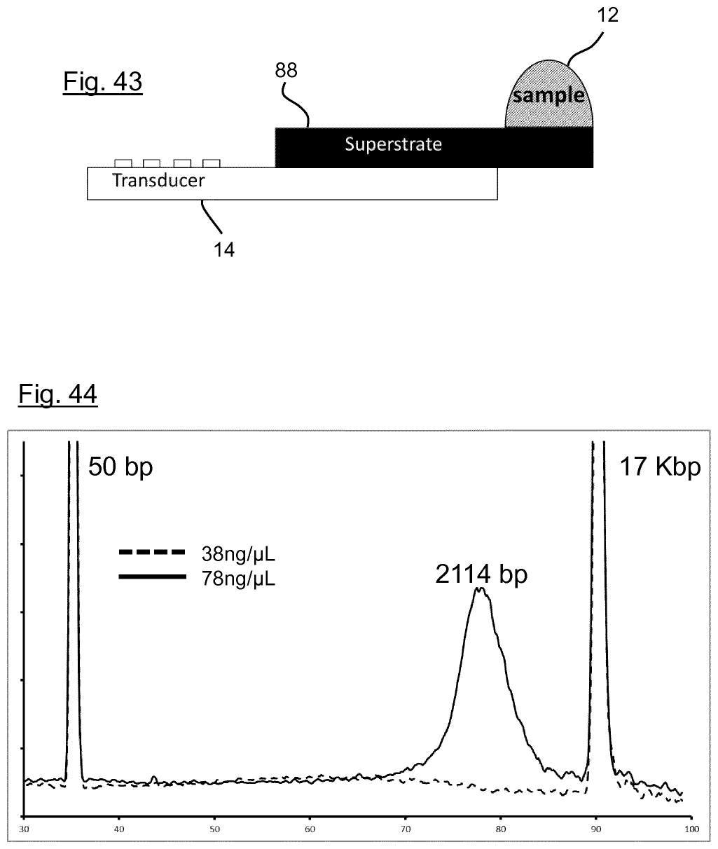

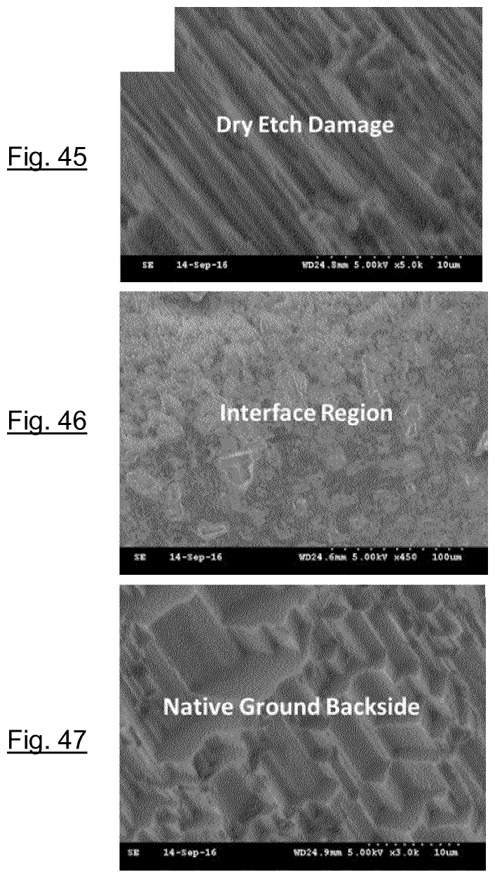

This dependence on acoustic power is shown in the fragmentation carried out on a flat silicon superstrate 88 (a piece of unprocessed silicon wafer), in the arrangement shown in FIG. 43. In this arrangement, it was not possible to stop the 9 μL sample 12 from moving to the far edge of the superstrate 88. The drop perched at the end where the surface displacement would be highest with respect to rest of the superstrate 88. With this arrangement peak fragment size distributions of around 2000 bp can be produced, as shown in FIG. 44. In FIG. 44, fragmentation size distributions are plotted for two concentrations of DNA, after a 7.3 MHz SAW with a power of 10 W for 120 s was applied to the superstrate.

As shown in FIG. 44 compared with FIGS. 40, 42 and 35, the peak fragment sizes are larger on the flat superstrate than that obtained by using structured silicon. However, although may not be desirable to have a drop perched on the end of the superstrate, the result informs us that structures designed to manipulate the acoustic field allows the reduction of the applied power and yet still induce useful fragmentation of DNA.

For the superstrate reported in FIG. 44, the surface roughness was gauged by white light profilometry (ContourGT Bruker), giving a measure the surface roughness of about Rz=800 nm.

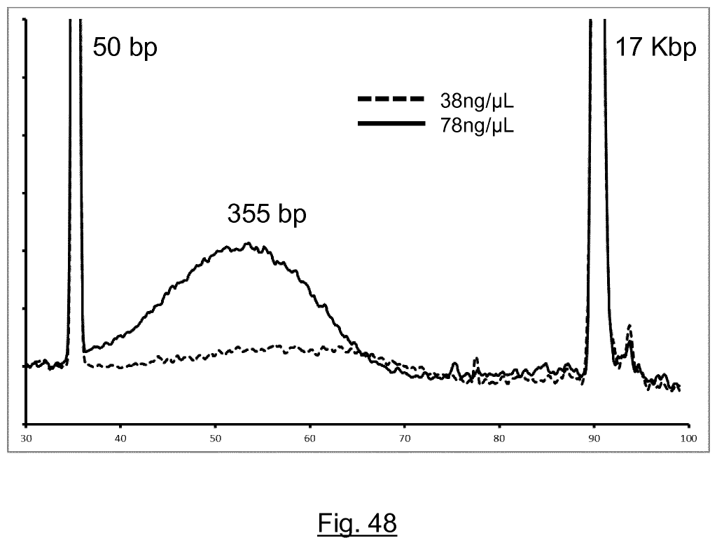

It was initially considered by the inventors that that the structures needed to be ordered in order to be of benefit to DNA fragmentation performance. However, a silicon wafer that has been used as a backing wafer for a dry etch process develops an altered texture on the backside of the wafer at its periphery. The measured roughness (gauged by white light profilometry (ContourGT Bruker)) of this area of the wafer was about Rz=19 μm. FIGS. 45-47 show SEM images of different parts of the Si superstrate. Fragmentation of DNA was carried out by placing the sample on the dry etch damages area and applying SAWs. It was found that this provided the best results for fragmentation without the need to have the sample temperature kept below 5° C. This is shown in FIG. 48, which shows the fragmentation size distribution for two concentrations of DNA, after exposure to 7.3 MHz SAW with a power of 10 W for 120 s, where a fragmentation peak of 355 bp was obtained for the higher concentration.

Comparison of FIGS. 44 and 48 illustrates the effect of non-ordered surface roughness on the DNA fragment size distribution.

SU8 is a negative photoresist that crosslinks a monomer after exposure and baking prior to development in EC solvent. The permanent crosslinking of the monomers in the resist provide resilient structures that will not completely deform when heated. Some reflow of the resist is expected when undergoing hard bakes (120° C. to 230° C.) as complete cross linkage occurs at 240° C. This ability of the polymeric coating to undergo plastic deformation was used to fabricate smooth micron scale structures.

Two formulations of SU8 were used (designed 3050 and 3025). These respectively provided layer thickness of about 45 μm and 20 μm. In order to obtain different sidewall angles the 3050 samples were exposed at 30 s, 120 s, 600 s and 900 s. After development of the optical lithography the various samples were hard baked at 120° C., 180° C. and 230° C. between 3 hrs and 20 hrs.

Higher temperature or longer times for the hard bake causes either more reflow or smoothing of the surface of the SU8.

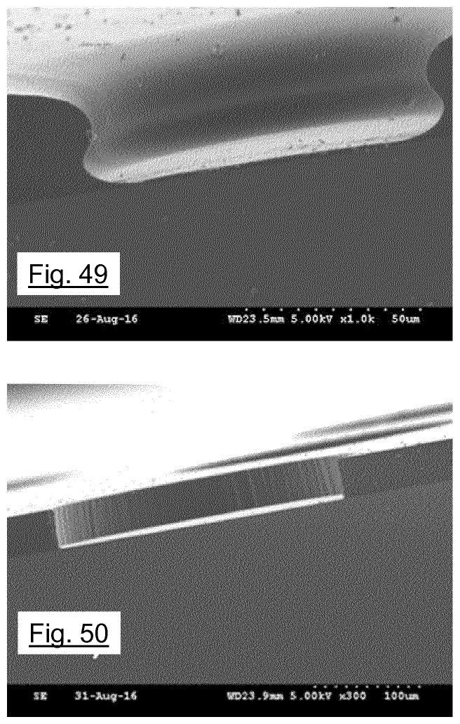

FIG. 49 shows an SEM cross sectional perspective micrograph of a SU8 3050 structure in the form of 100 μm diameter pits in a square array with 0.5 mm pitch. The structure was subjected to a 10 min exposure and subsequently a 230° C. 20 hrs hard bake. Note the smooth sidewalls and undulating shape and a thin layer approximately 1 μm thick on the silicon superstrate inside the pit.

FIG. 50 shows an SEM cross sectional perspective micrograph of a SU8 3050 structure in the form of a 300 μm pit. The structure was subjected to a 30 s exposure and subsequently a 120° C. bake for 4 h.

The structures in FIG. 49 did not produce DNA fragments using 13 W applied power. The structures in FIG. 50 showed little if any fragmentation of DNA. It is possible perhaps that this inefficiency could be attributed to the thin residual SU8 layer found in these structures. This may have hindered transmission of acoustic energy to the sample or that the SU8 coating hindered the transmission of acoustic energy to the sample. Compared will the pillar embodiments described later, it is possible that these structures have more SU8 covering the silicon, affecting their performance.

There were also manufactured arrays of pillars using SU8. Pillars with a diameter of 0.3 mm and pitch of 0.5 mm appeared to be less efficient than similar structures fabricated wholly in silicon.

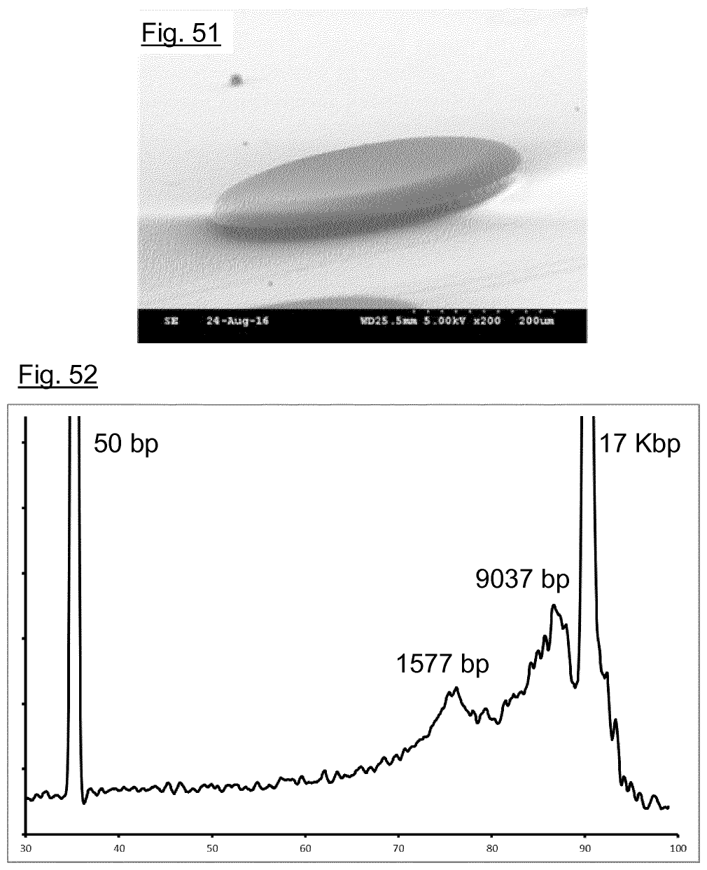

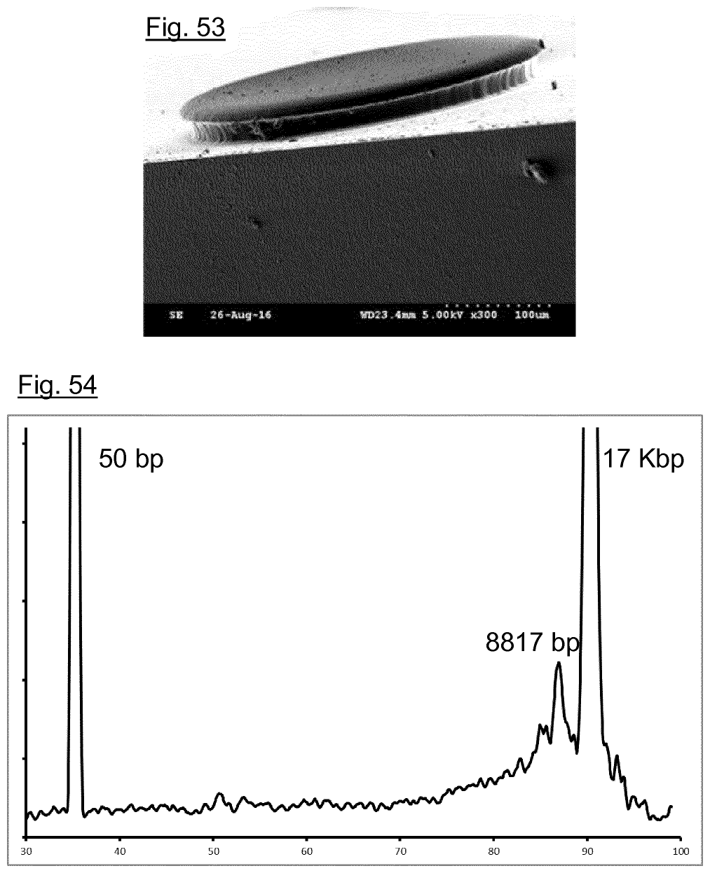

FIG. 51 shows a perspective SEM micrograph of an SU8 3050 pillar after 120 s exposure and 230° C. hard bake for 3 hrs. FIG. 53 shows a perspective SEM micrograph of an SU 8 3050 pillar after 600 s exposure and 230° C. hard bake for 20 hrs. Both structures were 0.3 mm in diameter and were fabricated in a square array formation with a pitch of 0.5 mm.

FIGS. 52 and 54 respectively show the performance of the structures of FIGS. 51 and 53 for DNA fragmentation. Each structure appeared inefficient for the fragmentation of DNA (43 ng/μL Promega) using 13 W applied power at approximately 7.3 MHz for 120 s. There also appeared to be some influence of the hard bake temperature and times used where higher temperature and longer time appear to lower the performance of the structures.

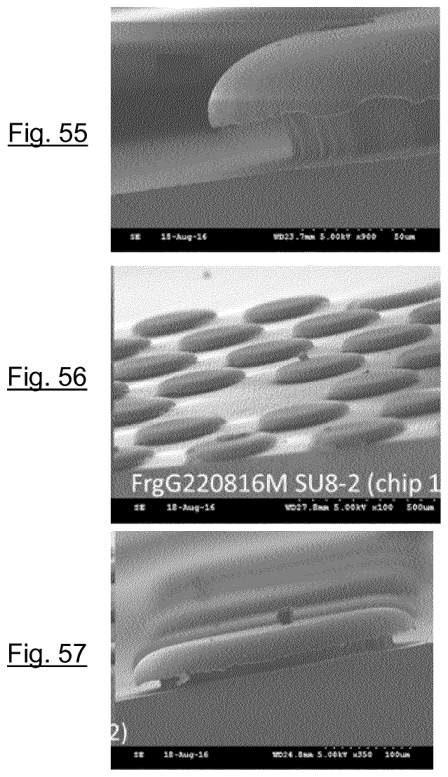

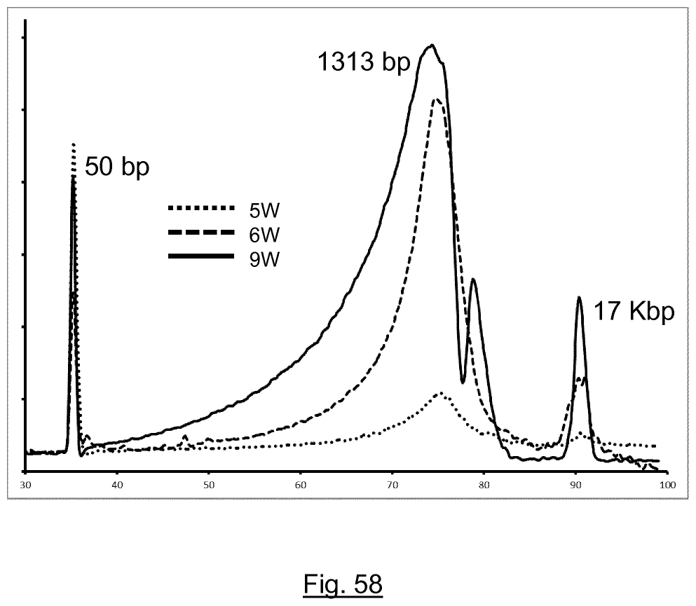

FIGS. 55-57 show SEM micrographs of other SU8 pillar structures. These were SU8 3050 exposed 900 s with a 3 hr hard bake at 180° C. The pillars were 0.3 mm in diameter placed in a square array with a pitch of 0.5 mm.

FIG. 58 shows plots of DNA (43 ng/μL Promega) fragmentation after exposure to 7.3 MHz SAW for 120 s with different applied powers, placed on an array of pillars as shown in FIGS. 55-57. The plots do not provide any clear trend with applied power but imply an optimal applied power for a particular engineered structure. However, it is clear that the presence of the pillars do enable fragmentation to occur at applied powers much lower than 13 W with a high yield for fragmented DNA.

Thus, some pillar structures (having the same diameter of 0.3 mm and pitch of 0.5 mm) perform better than others, comparing FIG. 58 with FIGS. 54 and 52. In FIG. 58, peak fragment sizes of approximately 1313 bp were obtained at relatively low applied power (5 W) and where the sample temperature was kept at approximately 18° C.

It is possible to think of the array of pits as a continuous layer of SU8 on the silicon superstrate whereas an array of pillars can be considered a discontinuous layer of SU8. The mass loading on the silicon superstrate will be lower in the case of a discontinuous coating. This may be an additional reason for the poor performance of the pit structure devices compared to the good performance of the pillars structures.

SU8 device structures were manufactured that were 1D arrays of troughs or strips. These were formed using similar conditions to the structures shown in FIGS. 55-57. The arrays were placed such that they were normal to SAWs produced by the transducer. That is, the arrays were parallel to the electrodes of the IDT. Here we see that the sidewall of the SU8 structure influences the efficiency of the device for fragmentation.

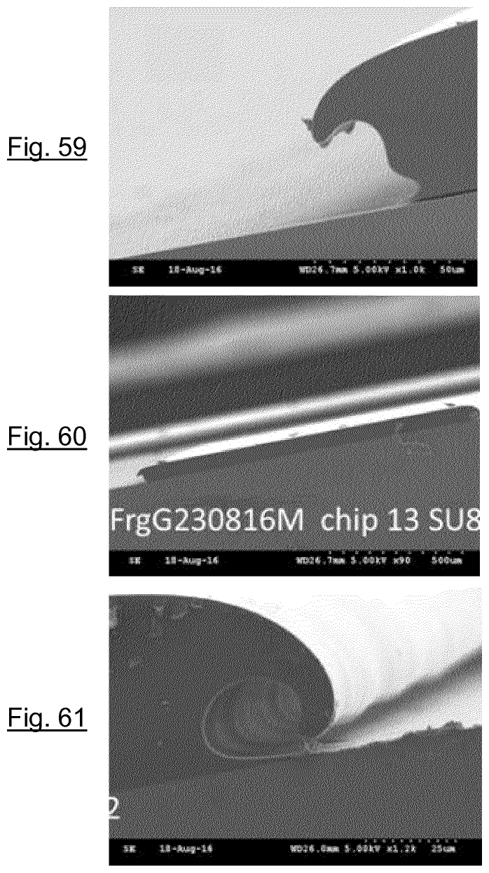

FIGS. 59-61 show SEM micrographs of SU8 3050 strip structures exposed for 900 s and hard baked at 180° C. for 3 hrs.

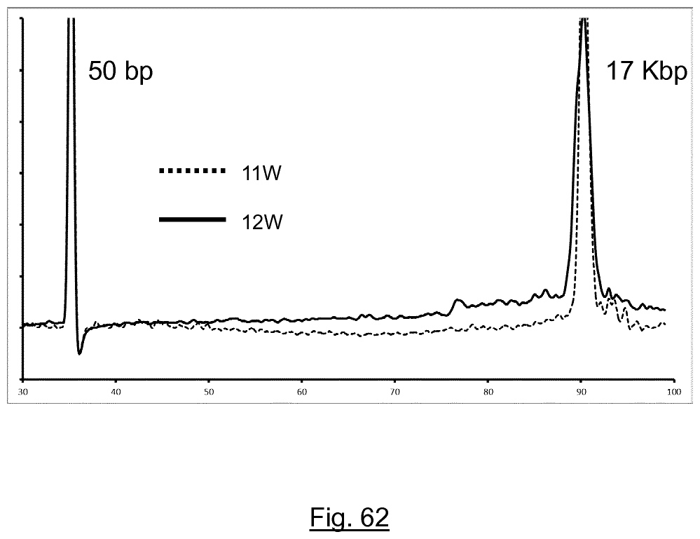

FIG. 62 shows the DNA fragmentation performance of the structures of FIGS. 59-61, carried out on a 9 μL sample of 43 ng/μL (Promega) DNA at about 7.3 MHz at 11 W applied power for 120 s. Raising the applied power to 12 W gave some improvement. However, the undulating or sigmoidal profile of the side wall of the strip structure shown in FIGS. 59-61 appears to inhibit efficient fragmentation of DNA using SAW.

However, it is found that if the sidewall is linear and an angle of approximately 60° fragmentation could be achieved at 11 W.

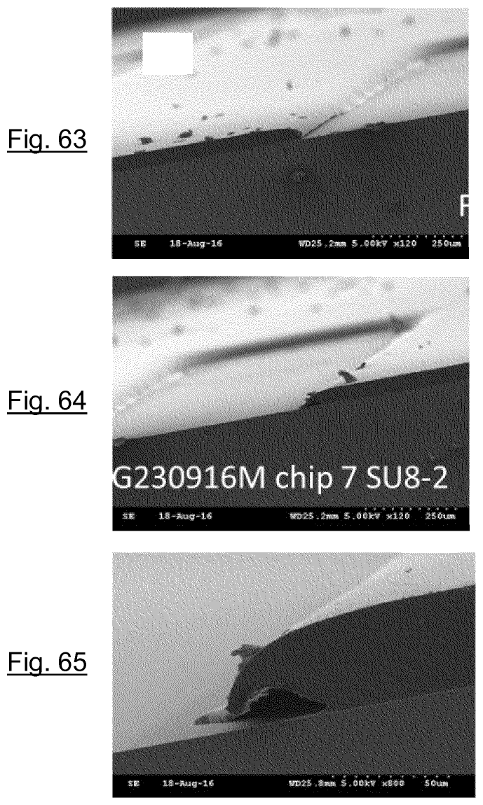

FIGS. 63-65 show SEM micrographs of SU8 3050 trough structures which were formed by exposing for 900 s and hard baking for 3 hrs at 180° C.

It was found that the strip structures performed much better than their trough structure counterparts. SU8 strip structures 1 mm wide with a pitch of 4 mm appeared to perform the best while strip structures 0.5 mm wide and a pitch of 2.5 mm also gave good results. The SU8 3050 layers were approximately 45 μm thick whereas the SU8 3025 layers were approximately 20 μm thick. There appeared to be a slight bias towards a smaller step height of the structures with respect to yield of fragments whereas peak fragment size appeared to show the reciprocal relationship where bigger step height produced fragment distributions peaks with lower base pair number.

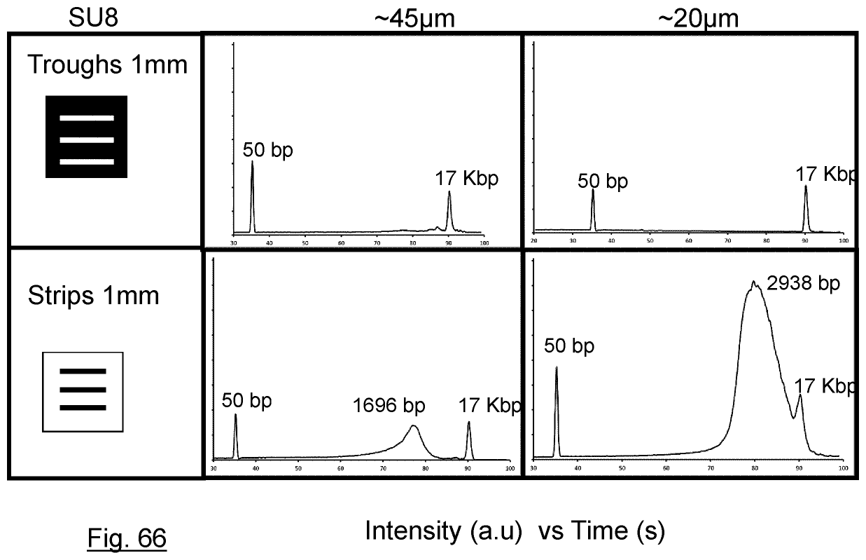

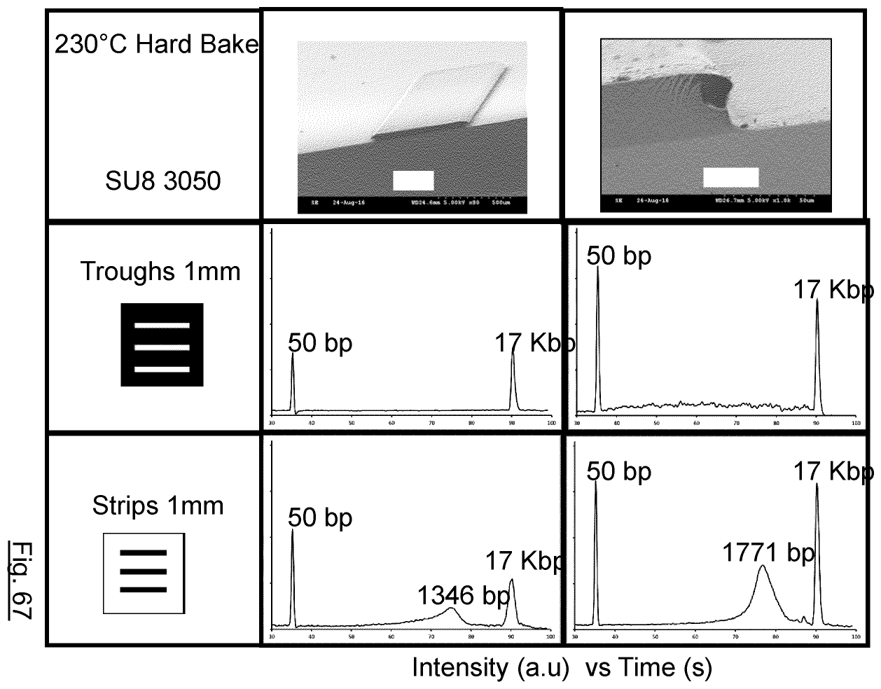

FIG. 66 shows the superior performance of strips with respect to their reciprocal structure (troughs) when a 9 μL sample (43 ng/μL Promega) is exposed to approximately 7.3 MHz with an applied power of 13 W for, in the case of the troughs 120 s, in the case of the strips 60 s. Note that the SU8 3050 device gave smaller peak fragment size while the SU8 3025 device had a higher yield. All devices used were hard baked at 120° C. for 4 hrs.

It was noticed that hard bake temperature and time influenced the performance of the SU8 devices. Devices that underwent a long 230° C. hard bake performed less well with respect to yield of material. This would imply that the degree of cross linking of the resist and hence elastic properties changes may be the cause of the difference.

In FIG. 67, a high temperature hard bake for different time periods are compared, where sample of 9 μL volume were used exposed to 7.3 MHz with an applied power of 13 W, samples on the trough structures were fragmented for 120 s, while samples on strip structures were fragmented for 60 s. There is some increase in sidewall curvature for the longer baked devices as can be seen from the SEMs in FIG. 67 but this appears small. Again the trough devices perform poorly compared to their strip counterparts. Both strip devices gave fragment distributions below 2 kbp for the conditions used.

In more detail, FIG. 67 compares SU8 3050 strip and trough structures that have undergone different hard bake times at 230° C. The SEMs for the 20 hr device exhibited slightly more curvature of the sidewall. Approximately 7.3 MHz with an applied power of 13 W was used to fragment 9 μL samples (43 ng/μL Promega) in the case of the troughs 120 s, in the case of the strips 60 s. Again troughs did not perform well whereas strip structures gave a high yield with fragment distribution peak below 2 kbp.

We now explain in general terms how the embodiments of the invention can be integrated into a DNA sequencing process, for implementation for example using a sequencing apparatus.

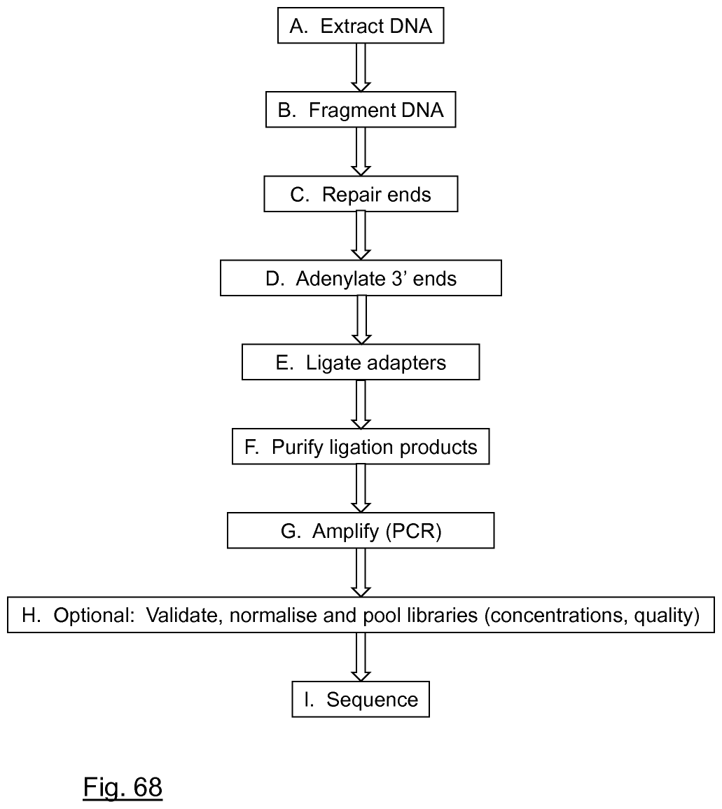

There are known to the skilled person various approaches for DNA sequencing. FIG. 68 illustrates a series of step for treatment of a DNA sample before the actual sequencing operation is carried out.

At step A, DNA is extracted. This step is dependent upon the nature of the sample, and may require lysis and/or purification.

At step B, the DNA is fragmented. This is carried out as explained in detail above.

After fragmentation, usually the fragment ends have overhangs that need to be blunted (using enzymes). This is done at step C.

At step D, an A is added at the end to provide an anchor for the adapter and prevent concatenation during ligation.

At step E, adapters (short DNA fragments) are added at the end of the sample DNA fragments to enable their hybridization onto the sequencing surfaces.

At step F, the adequate constructs are selected using size.

At step G, because the amount of adequate DNA constructs at this stage can be limited, a PCR amplification step is carried out. This also has the effect of purifying the sample.

Step H is optional. Here, there is the step of validating, normalising and pooling libraries (concentrations, quality).

Next, at step I, the pre-processed sample is subjected to the DNA sequencing operation itself.

One or more of steps C to H may be carried out at the sample treatment device used to carry out step B. Alternatively, the sample may be moved using either robotics (pipetting) or microfluidics (pressure or EWOD (electrowetting on demand), for example).

There are now described approaches to the fragmentation of DNA using freeze-thaw cycling methods, without necessary applying SAWs or other acoustic waves to the sample. However, it is to be understood that these freeze-thaw methods may be used in combination with the SAW or acoustic wave methods set out above.

Considering first the background to freeze-thaw treatment of DNA, it is known that multiple freezing and thawing of stored sperm samples will cause degradation of chromosomal DNA [Kopeika et al (2015)]. This is a method that has been used to increase the efficiency of gene modifications in gametes [Ventura et al (2009)]. In most cases however, fragmentation of DNA from freeze-thawing is a negative effect that methods have been designed to avoid. For example, in mass spectrometry, samples are frozen and desorbed from a frozen state (without thawing) to avoid unwanted fragmentation, as disclosed in EP-B-0404934 and U.S. Pat. No. 4,920,264.

Freeze-thawing has been used in preparing libraries for DNA sequencing, but the control obtained on the size or the efficiencies have not made it a preferred methodology [Makarov and Langmore (1999)]. Indeed the sizes of specific protocols have been characterised and shown to be higher than 10 kb [Shao et al (2012)], too large for efficient library preparation.

EP-A-1752542 discloses a method of generating non-human transgenic animals, including a freeze thaw step to cause fragmentation. U.S. Pat. No. 4,920,264 discloses a method for preparing samples for mass analysis by desorption from a frozen solution. One of the stated aims of this document is to mitigate or minimize fragmentation, and aims to achieve this by freezing target molecules. U.S. Pat. No. 6,117,634 discloses nucleic acid sequencing and mapping and mentions fragmentation as a by-product of freeze-thawing but does not utilise this. WO 2011/031127 discloses a method of isolating DNA from cells, in which rapid freeze thaw cycles between −65° C. and 70° C. are used.

Here we provide a platform, preferably based on a structured surface, to enhance the efficiency of the fragmentation of DNA by using temperature cycling, which includes a step below the freezing temperature of water. Note that in some circumstance it would be possible to freeze a sample above the triple point of water by the addition of a suitable additive. See, for example, http://news.mitedu/2016/carbon-nanotubes-water-solid-boiling-1128 [accessed 10 Oct. 2017].

The freeze-thaw method is illustrated here through the use of different heating mechanisms. The efficiency of the method appears to be linked to the presence of a microstructured surface (with feature size in the range of tens of microns, as described above).

In the work described below, human genomic DNA obtained from Promega Corporation (G3041) and Coriell NA12878 DNA was used with a concentration of 43 ng/μL and 38 ng/μL respectively, where each sample had a volume of 6 to 9 μL.

Two superstrates were used: a glass coverslip and structured silicon (consisting of pillars 130 μm in diameter, 160 μm high with a pitch of 230 μm) physically connected to a heat source via a small volume of heatsink compound while some samples were carried out directly on PZT\SU8 composite devices (see below).

The heaters were also thermally connected to a Peltier cooler rated for 6 A via a small volume of heatsink compound.

The power applied to the heaters was modulated such that the drop could melt and refreeze during each cycle of the modulated applied power, enabling multiple freeze/thaw cycles. Typically the modulation consisted of a square wave with a frequency between 0.05 Hz to 0.5 Hz.

In a first approach, an RF heater was used. Lead Zirconate Titanate (PZT) is a ferroelectric ceramic which can be used in the fabrication of piezoelectric transducers. A composite material was formed, comprising Ferroperm Pz26 powder (PZT) added to

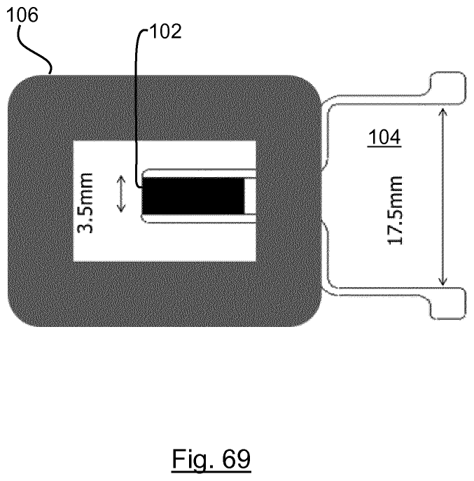

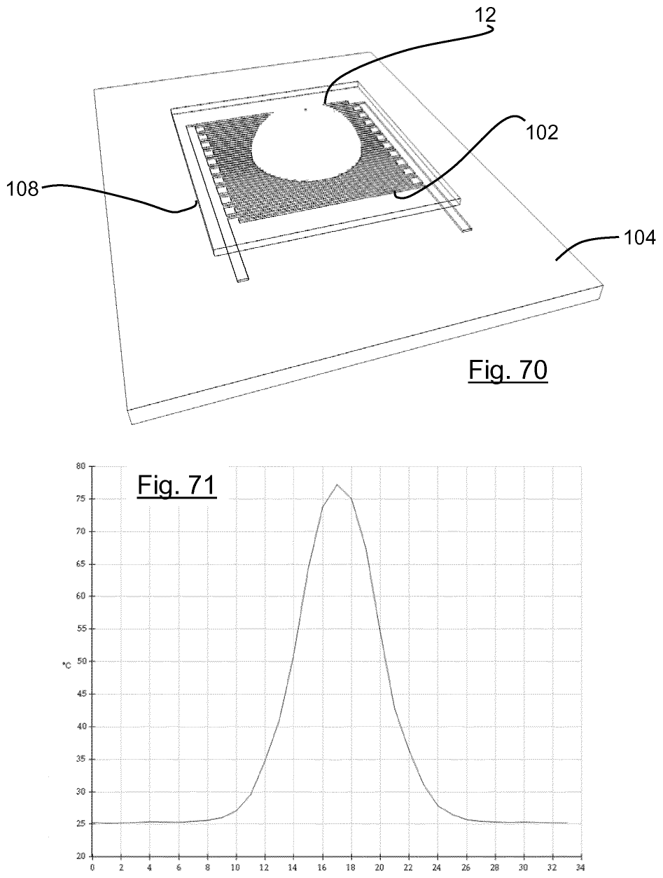

SU8 2050 negative tone photoresist at 30% by volume and mixed thoroughly. The mixture was then applied to interdigitated electrodes [obtained from Epigem UK] by first masking off the interdigitated electrodes with Sellotape®, with the excess mixture scraped off using the edge of a glass slide. This is illustrated in FIG. 69, showing the interdigitated electrodes 102 on SU8 coated glass 104. An area over the interdigitated electrodes 102 was masked off using Sellotape® 106. This area was then coated in the 30% by volume mix of PZT powder dispersed in SU8 photoresist. Excess applied mixture was scraped off leaving a film approximately 150 μm thick after processing (corresponding to the thickness of the Sellotape®). After pre baking the mixture at 95° C. for 20 min the device was exposed to 365 nm UV (23 mW/cm²) for 10 min then post exposure baked for 10 min at 95° C. in order to crosslink the photoresist.

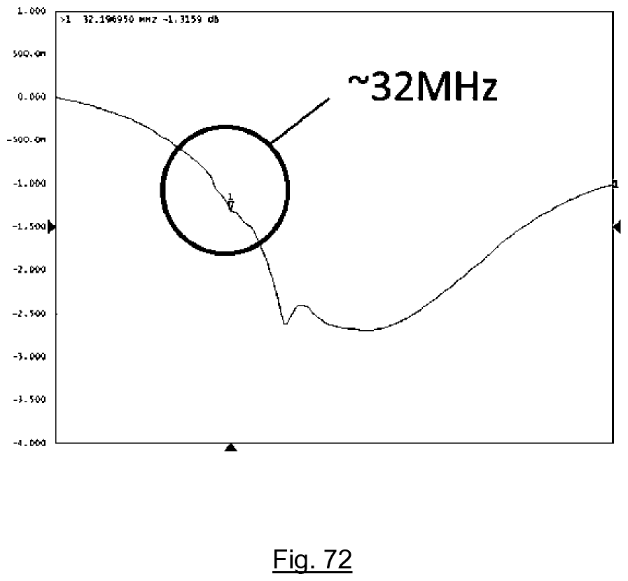

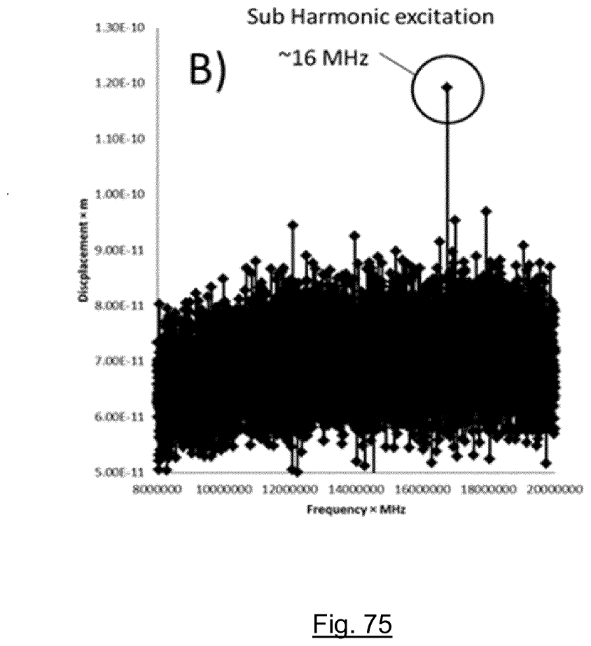

In the present approach, we do not use the PZT/SU8 composite devices for their piezoelectric properties, but for their roughness and ability to heat up quickly on application of a suitable RF signal via the electrodes. To avoid any doubt, we include a characterisation in FIG. 72 and in FIG. 75.

FIG. 72 shows a frequency scan of the PZT/SU8 composite material on an Epigem interdigitated electrode using an Agilent vector network analyser (S11 parameter). Marked on the scan is a small trough indicative of resonance point around 32 MHz which, although very much smaller than may be expected, is the correct frequency for the device.

FIG. 75 shows a polytec GmbH single point vibrometer (range up to 24 MHz) showing the presence of the first sub harmonic due to the restricted range of the vibrometer used (up to 24 MHz) when excited by a 5V pkpk signal at 32 MHz, indicating some actuation of the surface.

FIGS. 72 and 75 therefore show very small piezoelectric actuation of the surface, which is considered not to be significant enough to contribute meaningfully to a DNA fragmentation process.

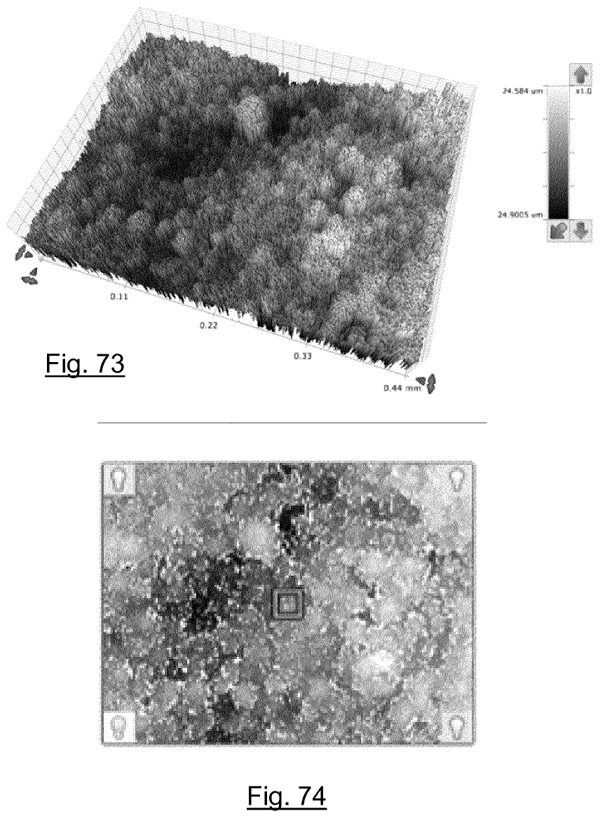

FIGS. 73 and 74 show screen shots from a Bruker Contour GT white light profilometer scan of the surface of the 30% by volume mix of PZT/SU8 composite. The surface is apparently non smooth appearance. Based on these results, the average roughness was found to be about 5 μm.

Heating using RF was found to be efficient where only 0.1 W of applied power at 32.5 MHz was enough to obtain a temperature of approximately 77° C. after 5 s. FIG. 71 shows a temperature plot across the central part of the device corresponding to the centre of the electrodes 102. This was plotted from an IR image taken with a FLIR IR camera of the PZT/SU8 composite device driven at 32.5 MHz with an applied power of 0.1 W for 5 s. The hottest part of the image is situated at the centre of the interdigitated electrodes. The maximum measured temperature was 77° C., with a temperature of at least about 44° C. measured at all areas directly above the interdigitated electrodes.

FIG. 71 shows a schematic perspective view of the device in operation. Base 104 holds the interdigitated electrodes 102 and the PZT/SU8 composite 108 is formed over and in contact with the interdigitated electrodes 102. Sample 12 has a volume of about 9 μL in this example.

Note that if the PZT/SU8 composite 108 was not present, no significant heating effect was observed when the RF signal was applied to the electrodes 102.

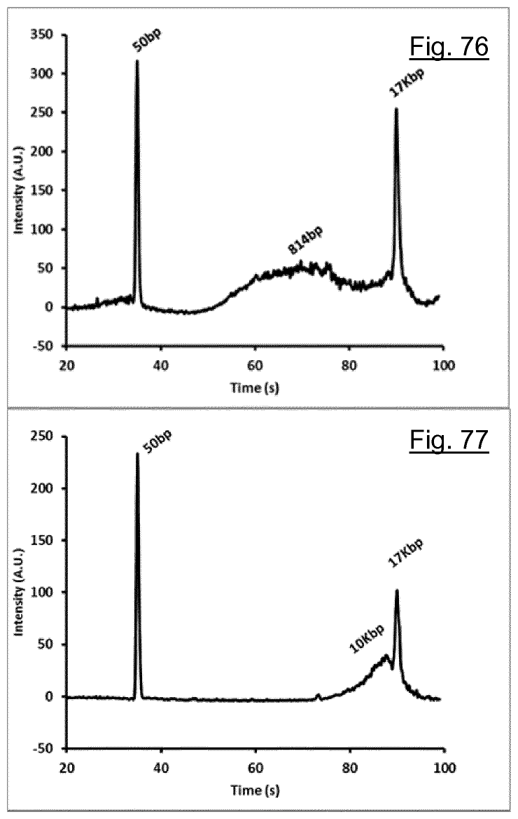

FIGS. 76 and 77 show electrographs of 9 μL of Human DNA (Coriell NA12878) samples each with a concentration of 38 ng\μL placed onto a PZT\SU8 composite device. In FIG. 76, the sample was in direct contact with the PZT\SU8 composite device. In FIG. 77, the sample was in contact with a smooth glass superstrate which itself was in direct contact with the PZT\SU8 composite device. The device was driven at 32.5 MHz with 0.2 W applied power modulated at a frequency of 0.05 Hz for FIG. 76 and driven at 32.5 MHz with 0.3 W applied power modulated at 0.05 Hz for FIG. 77. The starting temperature for FIG. 76 was −7° C. with the temperature ranging between −6° C. to 1° C. on the application of the RF signal which was applied for 8 min. The staring temperature for FIG. 77 was −6° C. with the temperature ranging between −5° C. to 1° C. on application of the RF signal which was applied for 5 min.

Based on FIG. 76, it can be seen that when cycling between solid and liquid states of water, DNA fragments below 1 kb can be obtained, when the sample is positioned directly on the rough surface (FIG. 76), while they do not form when the surface is smooth (FIG. 77).

Heating via a resistive heater was also found to be effective. Resistive thermal devices (RTD) were used as micro strip heaters. These devices were produce via a lift off technique, where the device pattern was created lithographically on 300 μm thick Pyrex glass followed by evaporating 100 nm of a suitable metal (e.g. Pt or NiCr) to create a serpentine track. The single wire was used as a heating element when a current, as high as 2.6 A, was passed through the device. The current supplied to the heaters was modulated with frequency of 0.05 Hz. The devices were used in conjunction with a superstrate. The superstrate was either a smooth glass coverslip or a structured silicon superstrate. These were coupled to the heater with the aid of heatsink compound.

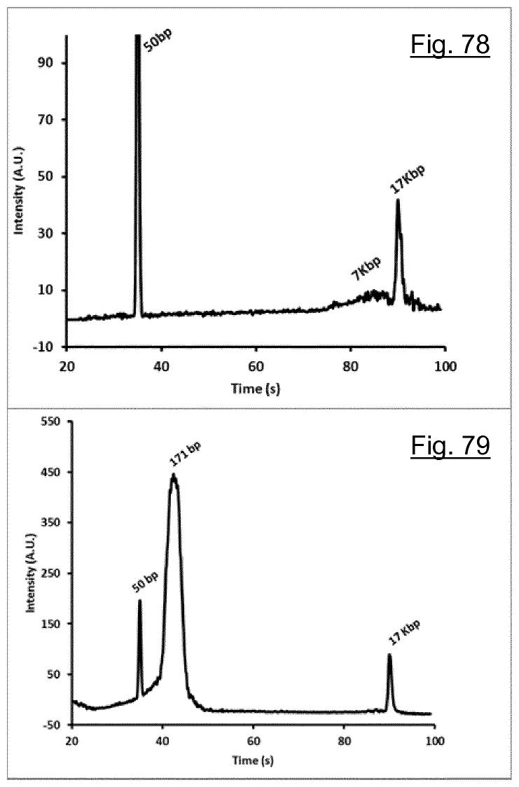

FIGS. 78 and 78 show electrographs of 6 μL of Genomic DNA (Promega G3041) samples each with a concentration of 43 ng/μL after treatment by heating with a micro strip heater. In FIG. 78, the sample was placed onto a coverslip (smooth glass superstrate). In FIG. 79, the sample was in contact with a structured silicon superstrate (pegs 130 μm dia. 160 μm high with a pitch of 230 μm). The starting temperature for FIG. 78 was −7.3° C. with the temperature ranging between −2.1° C. to 6.7° C. on the application of the modulated dc current (0.05 Hz) applied for 4 min. The staring temperature for FIG. 79 was −7° C. with the temperature ranging between −3.2° C. to 4.7° C. on application of the modulated dc current (0.05 Hz) applied for 4 min.

It can be seen when comparing FIGS. 78 and 79 that only comparatively large fragments are produced when the sample is placed on a glass slide. However, there is a striking contrast with the structured superstrate, in which very short fragments (which are desirable) are produced with the silicon pillars.

The embodiments of the freeze-thaw approach tested here use open systems. However, further embodiments would adopt features disclosed with the respect to the SAW and acoustic wave embodiments. In particular, it is envisaged to use closed chambers and/or two phase enveloped samples. It is considered that the fragmentation efficiency found using the structured superstrate would follow the same behaviour found with respect to the dimensions and shapes of the structured superstrates investigated in the SAW and acoustic wave embodiments.

Different approaches can be taken for heating the sample. For example, the sample may be heated (directly or indirectly) by radiation (such as an IR source (e.g. a flame) or IR/terahertz/visible diode or other radiation source, or laser), convection (warm air) or by contact with a heated source.

It is also possible to include particles in the sample (e.g. magnetic beads or plasmonic particles) that preferentially would heat up upon activation (e.g. via radiation or other excitement). Such an approach may have additional advantages in the sense of providing additional surfaces for fragmentation. Furthermore, such an approach may provide a means for capture of fragments. For example, the beads may be coated with suitable capture molecules or surface. Note that in some embodiments it is preferable not to include such further particles, in view of the desirability of removing such particles before sequencing, which therefore adds an additional processing step.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

All references referred to above and/or listed below are hereby incorporated by reference.

### LIST OF NON-PATENT REFERENCES

Marine R. et al., Appl. Environ. Microbiol., 77 (2011), 8071
Q. Tseng, A Lomonosov, E. Furlonga and C. Merten “Fragmentation of DNA in a sub-microliter microfluidic sonication device” Lab Chip, 2012, 12, 4677-4682, DOI: 10.1039/C2LC40595D
Y. Okabe and A. Lee “LCAT DNA Shearing” Journal of Laboratory Automation, 2014, Vol. 19(2) 163-170
Larguinho M., Santos H. M., Doria G., Scholz H., Baptista P. V., Capelo J. L. “Development of a fast and efficient ultrasonic-based strategy for DNA fragmentation” Talanta 81 (2010) 881-886
N. Nama, P. Huang, T. Huang, F. Costanzo “Investigation of acoustic streaming patterns around oscillating sharp edges” Lab Chip, 2014, 14, 2824
Wu, T. & Chang, I., 2005. Actuating and detecting of microdroplet using slanted finger interdigital transducers. Journal of Applied Physics, 98(2), 024903-7
Shao et al BIOPRESERVATION AND BIOBANKING Volume 10, Number 1, 2012, -10.1089/bio.2011.0016
Hedskog C. et al., PLOS One, 5 e11345 (2010)
R. J. Shilton, S. M. Langelier, J. R. Friend and L. Y. Yeo “Surface acoustic wave solid-state rotational micromotor” Appl. Phys. Lett. 100, 033503 (2012) http://dx.doi.org/10.1063/1.3676660
http://www.mlo-online.com/freeze-thaw-cycles-and-nucleic-acid-stability-whats-safe-for-your-samples.php
http://online.liebertpub.com/doi/pdf/10.1089/bio.2011.0016
Kopeika, J.; Thornhill, A.; Khalaf, Y. The Effect of Cryopreservation on the Genome of Gametes and Embryos: Principles of Cryobiology and Critical Appraisal of the Evidence. Hum. Reprod. Update 2015, 21 (2), 209-227 DOI: 10.1093/humupd/dmu063.
Ventura, O. M.; Gutierrez, A.; Motoliu, J. Method of Generating Non-Human Transgenic Animals. EP1752542 (A1), May 27, 2009.
Hunter, J. M.; Lin, H.; Becker, C. H. Cryogenic Frozen Solution Matrixes for Analysis of DNA by Time-of-Flight Mass Spectrometry. Anal. Chem. 1997, 69 (17), 3608-3612 DOI: 10.1021/ac9703764.
Makarov, V. L.; Langmore, J. Compositions and Methods for Analysis of Nucleic Acids. WO9839485 (A2), Feb. 25, 1999.
Shao, W.; Khin, S.; Kopp, W. C. Characterization of Effect of Repeated Freeze and Thaw Cycles on Stability of Genomic DNA Using Pulsed Field Gel Electrophoresis. Biopreservation Biobanking 2012, 10 (1), 4-11 DOI: 10.1089/bio.2011.0016.

1. A method of fragmenting chains of nucleic acids in a liquid sample, the method comprising:
providing a liquid sample comprising chains of nucleic acids;
providing a sample treatment device, the sample treatment device having a sample treatment zone;
contacting said sample with said sample treatment zone;
generating and propagating surface acoustic waves (SAWs) along a surface of the sample treatment zone, said SAWs coupling into the sample to cause fragmentation of said chains of nucleic acids in the sample.

2. The method according to claim 1 wherein the liquid sample has volume V, an area of an interface between the sample and the sample treatment zone being area A, wherein the ratio A/V is at least 1000 m²/m³.

3. The method according to claim 1 or wherein the sample treatment zone includes an area having roughness Rz at least 10 μm.

4. The method according to claim 1 wherein the sample treatment zone includes an array of cavities, being ordered or non-ordered, the cavities cumulatively containing at least part of the sample, optionally all of the sample.

5. The method according to claim 1 wherein the sample treatment zone includes an array of pillars, being ordered or non-ordered.

6. The method according to claim 1 wherein the contact angle between the sample and the sample treatment zone is lower than between the sample and a remaining part of the SAW transmission surface, in order to locate the sample.

7. The method according to claim 1 wherein the sample has a volume of not more than 30 μL.

8. The method according to claim 1 wherein the concentration of the chains of nucleic acids in the sample is in the range 5-100 ng/μL.

9. The method according to claim 1 wherein the SAW transmission surface is a surface of a superstrate coupled to the SAW transducer.

10. The method according to claim 1 wherein the temperature of the sample is controlled so as not to exceed 37° C.

11. The method according to claim 1 wherein the sample is subjected to active cooling.

12. The method according to claim 1 wherein the sample is frozen, or partially frozen, before the start of coupling SAWs into the sample.

13. The method according to claim 1 wherein, when the sample treatment zone is considered as the first sample treatment zone, the device includes an opposing member providing a second sample treatment zone, adapted to be located in contact with the sample opposite the first sample treatment zone, so that the sample is sandwiched between the first and second sample treatment zones, the opposing member being operable to be reciprocated relative to the SAW transmission surface.

14. The method according to claim 13 wherein, when the SAW transducer is considered as the first SAW transducer and the SAW transmission surface is considered as the first SAW transmission surface, the opposing member provides a second SAW transducer adapted to generate and propagate SAWs along a second SAW transmission surface including the second sample treatment zone, for coupling said SAWs into the sample to cause fragmentation of said chains of nucleic acids in the sample.

15. (canceled)

16. A sample treatment device for fragmenting chains of nucleic acids in a liquid sample, the device comprising:
a surface acoustic wave (SAW) transmission surface having a sample treatment zone;
a SAW transducer adapted to generate and propagate SAWs along the SAW transmission surface including the sample treatment zone, for coupling said SAWs into the sample to cause fragmentation of said chains of nucleic acids in the sample;
wherein the device includes an active cooling means in thermal contact with the sample treatment zone.

17. A sample treatment device for fragmenting chains of nucleic acids in a liquid sample, the device comprising:
a surface acoustic wave (SAW) transmission surface having a sample treatment zone;
a SAW transducer adapted to generate and propagate SAWs along the SAW transmission surface including the sample treatment zone, for coupling said SAWs into the sample to cause fragmentation of said chains of nucleic acids in the sample;
wherein when the sample treatment zone is considered as the first sample treatment zone, the device includes an opposing member providing a second sample treatment zone, adapted to be located in contact with the sample opposite the first sample treatment zone, so that the sample is sandwiched between the first and second sample treatment zones, the opposing member being operable to be reciprocated relative to the SAW transmission surface.

18. The sample treatment device according to claim 17 wherein, when the SAW transducer is considered as the first SAW transducer and the SAW transmission surface is considered as the first SAW transmission surface, the opposing member provides a second SAW transducer adapted to generate and propagate SAWs along a second SAW transmission surface including the second sample treatment zone, for coupling said SAWs into the sample to cause fragmentation of said chains of nucleic acids in the sample.

19. The sample treatment device according to claim 16 wherein one or more phononic structures are provided in order to affect the SAW distribution at the sample treatment zone.

20-78. (canceled)
